(19) United States
(12) Patent Application Publication
(10) Pub. No.: US 2026/0199412 A1
(43) Pub. Date: Jul. 16, 2026

(54) COMPOSITIONS AND METHODS COMPRISING NON-NATURALLY OCCURRING BACTERIA WITH IMPROVED PROBIOTIC FUNCTION AND LONG-TERM PERSISTENCE IN THE GREATER GUT

(71) Applicant: RUTGERS, THE STATE UNIVERSITY OF NEW JERSEY, New Brunswick, NJ (US)

(72) Inventors: Biju Parekkadan, Atlantic Highlands, NJ (US); Lorenzo Tosi, Flemington, NJ (US)

(73) Assignee: RUTGERS, THE STATE UNIVERSITY OF NEW JERSEY, New Brunswick, NJ (US)

(21) Appl. No.: 19/446,536

(22) Filed: Jan. 12, 2026

Related U.S. Application Data
(60) Provisional application No. 63/743,811, filed on Jan. 10, 2025.

(51) Int. Cl. A61K 35/747; A61K 35/00; C12N 1/20

(52) U.S. Cl. CPC A61K 35/747; C12N 1/20; A61K 2035/115

(57) ABSTRACT

Compositions and methods for the creation of bacteria having enhanced gut persistence are disclosed. Also disclosed herein are compositions and methods for the treatment of inflammatory gastrointestinal diseases such as Inflammatory Bowel Syndrome, Ulcerative Colitis, and Crohn's Disease.

### CROSS-REFERENCE TO RELATED APPLICATIONS

This application claims the benefit of the priority of U.S. Provisional Application No. 63/743,811, filed Jan. 10, 2025, the entire contents of which is incorporated herein by reference.

### FIELD OF THE INVENTION

The present invention relates to the generation of bacteria having desirable characteristics. More specifically the invention provides compositions and methods for the creation of bacteria having improved probiotic functions and enhanced gut persistence.

### BACKGROUND OF THE INVENTION

Several publications and patent documents are cited through the specification in order to describe the state of the art to which this invention pertains. Each of these citations is incorporated herein by reference as though set forth in full.

Lactobacillus is a well-known probiotic bacterium. In recent years, probiotics have become a multibillion-dollar industry due to reported health benefits including the stimulation of the immune system, the ability to aid in relieving constipation, and the production of important digestive enzymes. However, questions of efficacy have been raised due to the low survival rates and persistence in the gut of the probiotic following consumption. This in turn has led to different approaches being explored to improve the bioavailability and persistence of probiotics both during the journey into the gut, as well as within the gut.

Several groups have explored formulation and strain engineering techniques to improve survival rates and persistence of probiotics. Once such technique is encapsulating the probiotic, either in a food or biopolymer shells like alginate. However, this technique has a marginalized impact because of the inability to scale up the processes, varying levels of efficacy, costs, and lack of consumer acceptance of food-based encapsulation. Other known techniques have similar defects that prevent their widespread use. Accordingly, there is a clear need for improved methods and compositions for prolonging beneficial bacterial persistence in the digestive tract.

### SUMMARY OF THE INVENTION

In accordance with the present invention, methods for producing microorganisms with enhance gut persistence are provided. An exemplary method comprises administering the microorganism to a subject, collecting the fecal matter from the subject, and separating a modified microorganism from the fecal matter, said modified microorganism having enhanced gut persistence. In certain embodiments, the modified microorganism has a decreased elimination rate and/or increased bile salt resistance. In certain embodiments, the fecal matter is collected at least one day after administering the microorganism. In certain embodiments, the methods further comprise ex vivo culturing of the microorganism after separation from the fecal matter. In certain embodiments, the methods further comprise repeating steps a)-c) with the modified microorganism at least one time. In certain embodiments the microorganism is selected from L. rhamnosus, L. acidophilus, L. plantarum, L. rhamnosus, L. reuteri, L. casei, L. delbrueckii subsp. Bulgaricus, L. gasseri, L. fermentum, L. johnsonii, L. paracasei, L. salivarius, B. bifidum, B. animalis, B. breve, B. longum, B. adolescentis, B. infantis, Saccharomyces boulardii and Saccharomyces cerevisiae. In certain embodiments, the subject is a human or a mouse. In certain embodiments, the modified organism has a gut persistence that is at least 100% greater, at least 200% greater, or at least 300% greater than a control unmodified organism.

In certain embodiments, the modified microorganism has at least one persistence-associated genetic modification. In certain embodiments the at least one persistence-associated genetic modification is selected from the genetic modifications listed in Table 1, or is present in the genes listed in FIGS. 3B, 4, or Table 2.

In another aspect of the invention, microorganisms produced by any of the methods disclosed herein are provided. In certain aspects of the invention, probiotic formulations comprising these microorganisms are provided.

In another aspect of the invention, probiotic formulations comprising at least one microorganism having at least one persistence-associated genetic modification are provided. In certain embodiments, the at least one persistence-associated genetic modification is selected from the genetic modifications listed in Table 1 or is present in the genes listed in FIGS. 3B and 4. In certain embodiments, the microorganism has each of the persistence-associated genetic modifications present in the genes listed in FIGS. 3B and 4 or each of the genetic modifications listed in Table 1. In certain embodiments, the modified microorganism has a gut persistence that is at least 100% greater, at least 200% greater, or at least 300% greater than a control. In certain embodiments, the microorganism comprises a transgene. In certain embodiments, the transgene is selected from, GLP1, sTNFR1, IL-1RA, sGP130, EPO, PTH, insulin, or enfuvirtide and derivatives thereof.

In certain embodiments, the probiotic formulation comprises at least one formulary ingredient. In certain embodiments, the microorganism is present in a concentration of about 1×10⁶ CFU to about 1×10¹² CFU. In certain embodiments, the probiotic formulation comprises at least one anti-inflammatory agent. In certain embodiments, the anti-inflammatory agent is selected from corticosteroids, aspirin, celecoxib, diclofenac, diflunisal, etodolac, ibuprofen, indomethacin, ketoprofen, ketorolac, nabumetone, naproxen, oxaprozin, piroxicam, salsalate, sulindac, tolmetin, interleukin (IL)-1 receptor antagonist, IL-4, IL-6, IL-10, IL-11, IL-13, cytokine receptors for IL-1, tumor necrosis factor-alpha, IL-18 and derivatives and biosimilars thereof. Examples of inflammatory bowel disease therapeutics include, without limitation: anti-inflammatory agents, a TNF alpha inhibitor, monoclonal antibodies, a receptor fusion protein, steroids, aminosalicylates, and immunomodulators or immunosuppressants.

In another aspect of the invention, methods of treating an inflammatory disease comprising administering the probiotic formulations disclosed herein are provided. In certain embodiments, the methods further comprise administering at least one anti-inflammatory agent. In certain embodiments, the anti-inflammatory agent is selected from corticosteroids, aspirin, celecoxib, diclofenac, diflunisal, etodolac, ibuprofen, indomethacin, ketoprofen, ketorolac, nabumetone, naproxen, oxaprozin, piroxicam, salsalate, sulindac, tolmetin, interleukin (IL)-1 receptor antagonist, IL-4, IL-6, IL-10, IL-11, IL-13, cytokine receptors for IL-1, tumor necrosis factor-alpha, IL-18 and derivatives and biosimilars thereof. Examples of inflammatory bowel disease therapeutics include, without limitation: anti-inflammatory agents, a TNF alpha inhibitor, monoclonal antibodies, a receptor fusion protein, steroids, aminosalicylates, and immunomodulators or immunosuppressants. In certain embodiments, the inflammatory disease is selected from Crohn's Disease, ulcerative colitis, indeterminate colitis, lymphocytic colitis, ischaemic colitis, diversion colitis, microscopic colitis, infective colitis, collagenous colitis, Bahcet's syndrome, idiopathic inflammation of the small and/or proximal intestine, and IBD-related diarrhea. In certain embodiments, administering the probiotic composition ameliorates symptoms of the disease.

Still other aspects and advantages of these compositions and methods for making the compositions and using the compositions are described further in the following detailed description of the preferred embodiments thereof.

### BRIEF DESCRIPTION OF THE DRAWINGS

FIGS. 1A-1D: Repetitive autologous murine gastrointestinal tract gavage workflow and recovered CFUs mL-1 in daily fecal matter after gavage. FIG. 1A) Workflow of in-vivo evolution and selection of colonies. The average Logo of the CFUs mL-1 obtained from fecal matter after repetitive murine gastro intestinal tract gavage was plotted for each gavage (n=2). FIG. 1B) CFU counts from fecal matter obtained after gavage of wild type. The isolate collected from the end of gavage 1 is evolution 1. FIG. 1C) CFU counts from fecal matter obtained after gavage of evolution 1, which used colonies selected from gavage 1. The isolate collected from the end of the gavage of evolution 1 is evolution 2. FIG. 1D) CFU counts from fecal matter obtained after the gavage of evolution 2. Error bars were determined from standard deviation.

FIGS. 2A-2E: Extrapolated data from fecal collection after repetitive autologous murine gastrointestinal tract gavage. FIG. 2A) Values were extrapolated from the daily fecal collection for all three gavages various. These values include FIG. 2B) the average persistence time, FIG. 2C) the average elimination rate of the bacteria, FIG. 2D) the estimated bacteria totals for day 0 before fecal collection, and FIG. 2E) the difference between the gavage concentration of 0.5×10¹⁰ CFU mL⁻¹ and the sum of the total CFU mL⁻¹ from entire fecal collection period of each gavage. Error bars were determined from standard deviation.

FIG. 3A-3B: Breakdown of genetic variation types and gene families with recorded changes from next generation sequencing data. FIG. 3A) The legend indicates changes in the number of nucleic acid base pairs detected at each location from the original wild type genome to evolution 1. Labels show the number of variations of each type as well as total percentage of the total variations. From cross referencing the changes detected to the wild type genome FIG. 3B) a breakdown of gene categories, and number of genes changed in each category was determined.

FIG. 4: Single genes with more than 2 detected changes from next generation sequencing data.

FIG. 5A-5E: Growth curves and linear growth rates of wild type, evolution 1 and evolution 2 in bile salts in Various Concentrations of Bile Salt over 15 hours. Using a spectrophotometer, the absorbance at 600 nm was found for the three different variants every hour over a 15-hour span. The growth curve was plotted for the wild type and two evolutions in FIG. 5A) 0.75% bile salt and FIG. 5B) 1.0% Bile Salt. From these growth curves a breakdown of the time spent in each of the first three bacterial growth phases for both FIG. 5C) 0.75% and FIG. 5D) 1.0% Bile Salt, as well as FIG. 5E) linear growth rates of wild type, evolution 1 and evolution 2 in 0.5%, 0.75% and 1.0% bile salt in h⁻¹.

FIG. 6A-6C: Growth curves and linear growth rates of wild type, evolution 1 and evolution 2 in 0.5% bile salt over a span of 15 hours. FIG. 6A) Using a spectrophotometer, the absorbance at 600 nm was found for the three different variants every hour over a 15-hour span. The growth curve was plotted for the wild type and two evolutions in 0.50% bile salt (n=3). From these growth curves FIG. 6B) a breakdown of the time spent in each of the first three bacterial growth phases and FIG. 6C) linear growth rates of wild type, evolution 1 and evolution 2 in 0.50% bile salt in h⁻¹ are shown. Error bars were determined from standard deviation. ANOVA analysis shows differences between the groups was significant (*, p<0.05)

FIG. 7A-7C: Caco-2 binding perfusion workflow and recovered CFU mL-1 results. FIG. 7A) Workflow of Caco-2 Cell perfusion and bacteria collection. FIG. 7B) CFU mL-1 recovered from well plates after perfusion onto Caco-2 cells were plotted. The CFU counts taken from wells which were washed with PBS and then plated onto vancomycin plates (n=1) FIG. 7C) Percent adhesion of perfused bacteria onto Caco-2 cells, which is the total recovered CFUs divided by the total perfused CFUs.

FIG. 8A-8H: Heterologous gastrointestinal tract gavage recovered CFUs mL-1 in daily fecal matter after gavage and extrapolated data from heterologous fecal collection as compared to the repeated autologous gavages. FIG. 8A) The Log₁₀ of the CFUs mL-1 obtained from fecal matter after heterologous murine gastro intestinal tract gavages into a fresh mouse was plotted for each gavage (n=1). FIG. 8B) CFU counts from fecal matter obtained after the heterologous gavage of the wild type. FIG. 8C) CFU counts from fecal matter obtained after heterologous gavage of evolution 1. FIG. 8D) CFU counts from fecal matter obtained after heterologous gavage of evolution 2. The daily fecal collection for all three heterologous gavages were used to extrapolate FIG. 8E) the average persistence time, FIG. 8F) the average elimination rate of the bacteria, FIG. 8G) the estimated bacteria totals for day 0 before fecal collection, and FIG. 8H) the difference between the gavage concentration of 0.5×10¹⁰ CFU mL⁻¹ and the sum of the total CFU mL⁻¹ from entire fecal collection period of each gavage. Error bars were determined from standard deviation.

FIG. 9: Relationship between total accumulation of engineered secreted protein expression after 14 days of daily gavages and elimination rate of Lactobacillus rhamnosus from intestinal tract. This model uses a one-compartment, multi-dose administration to model the accumulation of bacteria in the gut, and with a constant secretion rate, the accumulation of a hypothetical ‘Drug X’ is plotted against the elimination rate of the bacteria which was varied from 0.2 to 2 Log₁₀ (CFU mL⁻¹) day-1. Figure was plotted using Matlab.

FIG. 10A-10D: Generation of evolved Lactobacillus rhamnosus comprising RFP. FIG. 10A) Culture of evolved Lactobacillus rhamnosus cells after Electroporation with 65 ng, 80 ng, and 100 ng of red fluorescent protein (RFP) plasmid DNA. FIG. 10B) Graphical representation of bacterial fluorescence. FIGS. 10C-10D) Fluorescence microscopy images showing uptake of the RFP plasmid DNA.

### DETAILED DESCRIPTION OF THE INVENTION

To overcome the challenges of the prior art, the invention described herein uses the principle of forced evolutionary engineering to generate non-naturally occurring bacteria with improved probiotic function and long-term viability by introducing natural stressors into the bacterial environment. In addition to forcing the bacteria to adapt to various extremes including temperatures and pH values, the methods described herein simulate complex in vivo conditions. Previous studies have not described forced evolution of a probiotic lactic acid bacteria through a mouse intestine to improve probiotic functional traits.

Based on the hypothesis that in vivo evolution of Lactobacilli through a process of in vivo oral administration, fecal selection, ex vivo culture, and re-formulation would yield strains with increased probiotic persistence and bioavailability, the following studies were performed. We report that Lactobacillus rhamnosus can be evolved through the aforementioned processes to quickly create new evolutionary strains with prolonged persistence and other in the digestive tract of mice. In vivo evolution was associated with genetic changes, bile salt resistance, and a lack of epithelial binding. These methods can be adapted to other stressors or disease models, to create evolved strains for the treatment of a variety of gut disorders.

The following definitions are provided to aid in understanding the subject matter regarded as the invention. Unless otherwise defined, scientific and technical terms used in connection with the formulations described herein shall have the meanings that are commonly understood by those of ordinary skill in the art The terminology used herein is for the purpose of describing particular embodiments only, and is not intended to limit the scope of the present invention, which is defined solely by the claims.

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e., combinations) of two or more of the compounds. According to the present invention, an isolated, or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

As used herein and in the claims, the singular forms, such as “a”, “an” and “the” include the plural reference and vice versa unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers. The term “or” is inclusive unless modified, for example, by “either”. The term “and/or” is intended to represent an inclusive or. That is “X and/or Y” is intended to mean X or Y or both, for example. As a further example, X, Y, and/or Z is intended to mean X or Y or Z or any combination thereof.

When ranges are used herein for physical properties, such as molecular weight, or chemical properties, such as chemical formulae, all combinations and subcombinations of ranges and specific embodiments therein are intended to be included. Other than in the operating examples, or where otherwise indicated, all numbers expressing quantities of ingredients or reaction conditions used herein should be understood as modified in all instances by the term “about”

The term “about” when referring to a number or a numerical range means that the number or numerical range referred to is an approximation within experimental variability (or within statistical experimental error), and thus the number or numerical range may vary between 1% and 15% of the stated number or numerical range, as will be readily recognized by context Furthermore any range of values described herein is intended to specifically include the limiting values of the range, and any intermediate value or sub-range within the given range, and all such intermediate values and sub-ranges are individually and specifically disclosed [e.g. a range of 1 to 5 includes 1, 1.5, 2, 2.75, 3, 3.90, 4, and 5]. Similarly, other terms of degree such as “substantially” and “approximately” as used herein mean a reasonable amount of deviation of the modified term such that the end result is not significantly changed. These terms of degree should be construed as including a deviation of the modified term if this deviation would not negate the meaning of the term it modifies.

The phrase “consisting essentially of” when referring to a particular nucleotide or amino acid means a sequence having the properties of a given SEQ ID NO: For example, when used in reference to an amino acid sequence, the phrase includes the sequence per se and molecular modifications that would not affect the functional and novel characteristics of the sequence.

### Methods for Producing and Screening Improved Microorganisms

The present invention provides methods for producing microorganisms, particularly Lactobacillus rhamnosus, having improved characteristics, such as gut persistence, decreased elimination rate, increased bile salt resistance or altered binding to Caco-2 cells. In certain embodiments, the methods include the steps of administering a microorganism to a subject, collecting fecal matter from the subject, and separating a modified microorganism from the fecal matter, thereby producing an improved microorganism. In certain embodiments, the fecal matter is collected at least one day after administering the microorganisms. In certain embodiments, the fecal matter is collected about 1-14 days after administering the microorganisms. In certain embodiments, the fecal mater is collected at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, or at least 14 days after administering the microorganisms. In certain embodiments, the methods further include ex vivo culturing the microorganism after separation from the fecal matter.

In certain embodiments, the methods comprise repeating these steps one or more times. In certain embodiments where the steps are repeated, the methods further include ex vivo culturing the microorganism after separation from the fecal matter and before readministering the modified microorganism to the subject.

In another aspect, the microorganism may have been previously genotyped and thus the genetic sequence and expression profile in the sample may be available. Accordingly, the method may entail storing reference sequence information in a database, i.e., those genetic alterations associated with a more favorable or less favorable phenotype as described herein, and performance of comparative genetic analysis on the computer, thereby identifying those microorganisms having improved gut persistence.

Genetically altered nucleic acid sequences associated with enhanced gut persistence, including but not limited to those listed below may be used for a variety of purposes in accordance with the present invention. Such nucleic acids can comprise DNA, RNA, or fragments thereof which may be used as probes to detect the presence of and/or expression of gut persistence specific markers. Methods in which gut persistence specific marker nucleic acids may be utilized as probes for such assays include, but are not limited to: (1) in situ hybridization; (2) Southern hybridization (3) northern hybridization; and (4) assorted amplification reactions such as polymerase chain reactions (PCR).

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single stranded or double stranded. The exact length of the probe will depend upon many factors, including temperature, source of probe and use of the method. For example, for diagnostic applications, depending on the complexity of the target sequence, the oligonucleotide probe typically contains 15 25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

Further, assays for detecting gut persistence genetic alterations may be conducted on any type of biological sample. Clearly, gut persistence genetic alteration containing nucleic acids, vectors expressing the same, gut persistence genetic alteration containing marker proteins and anti-gut persistence genetic alteration specific marker antibodies of the invention can be used to detect gut persistence genetic alterations in the microorganism, and alter gut persistence genetic alteration containing marker protein expression for purposes of assessing the genetic and protein interactions involved in the development of gut persistence.

In most embodiments for screening for gut persistence genetic alterations, the gut persistence genetic alteration containing nucleic acid in the sample will initially be amplified, e.g. using PCR, to increase the amount of the templates as compared to other sequences present in the sample. This allows the target sequences to be detected with a high degree of sensitivity if they are present in the sample. This initial step may be avoided by using highly sensitive array techniques that are important in the art.

Alternatively, new detection technologies can overcome this limitation and enable analysis of small samples containing as little as 1 μg of total RNA. Using Resonance Light Scattering (RLS) technology, as opposed to traditional fluorescence techniques, multiple reads can detect low quantities of mRNAs using biotin labeled hybridized targets and anti-biotin antibodies. Another alternative to PCR amplification involves planar wave guide technology (PWG) to increase signal-to-noise ratios and reduce background interference. Both techniques are commercially available from Qiagen Inc. (USA).

Any of the aforementioned techniques may be used to detect or quantify gut persistence genetic alteration specific marker expression and accordingly, identify microorganisms with increased gut persistence.

### Probiotics and Probiotic Compositions

The present invention provides probiotic compositions comprising at least one microorganism, particularly a Lactobacillus rhamnosus, having enhanced gut persistence when compared to a control non-evolved bacteria. In certain embodiments, the microorganism are prepared using the methods discussed above.

The terms “probiotic” and “probiotic preparation”, as may be used interchangeably herein, refer to a mixture of microorganisms which when orally administered can provide health benefits to a human or a non-human animal.

The term “probiotic formulation”, as used herein, refers to a formulation comprising a probiotic preparation formulated together with one or more additional formulary ingredients to obtain a finished formulation suitable for oral delivery to a human or a non-human animal.

In certain aspects of the invention, any microorganism with probiotic properties can be used. In certain embodiments, the microorganism is selected from a bacteria in the Lactobacillus, Bifidobacterium, Saccharomyces, Streptococcus, Enterococcus, Escherichia, or Bacillus genera. In certain embodiments, the microorganism is a Lactobacilli selected from L. rhamnosus, L. acidophilus, L. plantarum, L. rhamnosus, L. reuteri, L. casei, L. delbrueckii subsp. Bulgaricus, L. gasseri, L. fermentum, L. johnsonii, L. paracasei, and L. salivarius. In certain embodiments, the microorganism is a Bifidobacteria selected from B. bifidum, B. animalis, B. breve, B. longum, B. adolescentis, and B. infantis. In certain embodiments, the microorganism is a Saccharomyces selected from Saccharomyces boulardii and Saccharomyces cerevisiae. In one embodiment, the probiotic formulation further comprises a mixture of viable microorganisms independently selected from at least one, at least two, at least three, at least four, at least five, at least six, at least seven, at least eight, at least 9, at least 10, at least 11, at least 12, at least 13, at least 14, at least 15, at least 16, at least 17, or all 18 of the microbial species listed above.

The microorganism it can be grown in quantities, as desired, and cultured under appropriate conditions, for example in a liquid growth medium comprising appropriate microbial nutrients, and thereafter the microorganisms can be harvested under conditions ensuring that viable microorganisms are retained from the medium, and the harvested microorganisms can be used to prepare the probiotic formulations of the present disclosure. In embodiments hereof where the probiotic formulation comprises a plurality of microbial species, the species can be co-cultured, or alternatively separately grown and mixed upon harvesting.

In order to prepare the probiotic formulations of the present disclosure, a quantity of the microorganism is obtained and mixed to obtain a probiotic preparation.

In one embodiment, at least one microorganism is present in a concentration sufficient to prepare a probiotic formulation to a final concentration per dose of, for example, from about 1×10⁶ CFU to about 1×10¹² CFU for each species. In this manner, a probiotic preparation can be obtained. The probiotic preparation can then be used to prepare a probiotic formulation. Probiotic formulations as used herein are formulations comprising a probiotic preparation formulated together with one or more additional formulary ingredients. Upon formulation, the probiotic formulations of the present disclosure can be directly used for oral delivery, including in the form of a dietary supplement or medicinal food. Probiotic formulations further also include formulations that can be used as a formulation ingredient for inclusion in a food or a feed. The probiotic formulations of the present disclosure can for example, be incorporated in a dairy product, such as milk, and in particular a fermented dairy product, for example with yogurt ferments, or other food products such as a snack bar, or beverages, such as a fruit or vegetable juice. In further embodiments, the probiotics may be included in a medical food, such as medical foods intended for dietary intervention, for example medical food comprising extensively hydrolyzed casein formula supplemented with probiotic strains e.g. Lactobacillus rhamnosus, probiotics containing biscuit, or a milk-based fruit drink containing a probiotic strain, or rectally delivered as a suppository or enema.

Formulary ingredients that can be used to prepare a finished probiotic formulation can vary substantially. In one embodiment, the finished probiotic formulation, in addition to the viable microorganisms can include a formulary ingredient suitable for incorporation in a probiotic formulation selected from a binder, such as a starch, sugar or cellulose, or a derivative thereof; an excipient, such as gelatin, or polyethylene glycol; or a diluent, such as water, or a buffer, for example. Orally dosed formulations, for example, can, in addition to the viable microorganisms comprise, inert compression aids, such as microcrystalline cellulose or oligosaccharide, flow aids, such as a silica gel, or a lubricant of, for example magnesium stearate (vegetable source) or stearic acid (vegetable source). Suppository formulations, for example, either for rectal or vaginal use, can in addition to the probiotics, comprise, for example, cocoa butter, polyethylene glycol, glycerine or gelatine.

In one embodiment, the probiotic formulation can comprise a prebiotic as a formulary ingredient. In one embodiment, the prebiotic can be selected from the group of prebiotics consisting of fructooligosaccharides, P95 NUTRAFLORA® (soluble prebiotic fiber containing a minimum of 95% (dry basis) shortchain fructooligosaccharides), for example, galactooligosaccharides, xylooligosaccharides, isomaltooligosaccharides, human milk oligosaccharides, inulin oligosaccharides, mannan oligosaccharides, pyrodextrin, levan, maltotriose, pectic oligosaccharides, bimuno-galactooligosaccharides, arabinoxylan, and fucoidan.

Formulary ingredients can be contacted with a probiotic preparation and mixed or prepared until a probiotic formulation is obtained. As will be clear to those of skill in the art, formulation conditions will generally be such that viable microorganisms are retained. In particular high temperatures, for example temperatures in excess of 40° C. are avoided.

The probiotic formulations in accordance herewith can vary substantially and can include solid or semisolid formulations, as well as liquid formulations and can further include powders, tablets, such as lozenges or effervescent tablets, pastilles, or capsules.

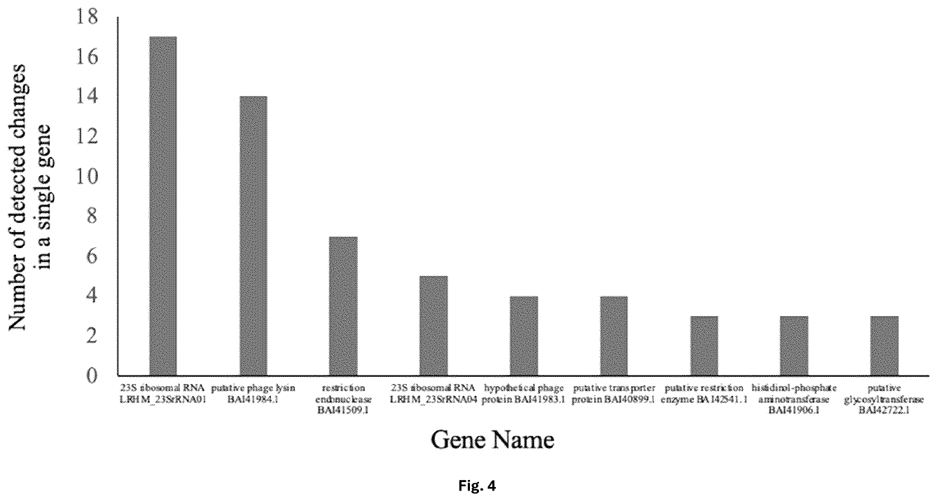

In certain embodiments, the microorganism comprises at least one gut persistence-associated genetic alteration. In certain embodiments, the genetic alteration is in a gene encoding a protein selected from a transferase, ATP-Binding protein, kinase, synthase, regulator protein, hydrolase, phage protein, ATPase/GTPase, Ribosomal protein/Ribosomal RNA, transporter protein, substrate-binding protein, permease, PTS system protein, Polymerase, cell surface protein, and reductase. In certain embodiments, the genetic alteration is selected from the genetic alterations listed in Table 1. In certain embodiments, the genetic alteration is in a gene listed in FIG. 4 or in Table 2.

TABLE 1

| #CHROM | POS | REF | ALT | Gene Type |
|---|---|---|---|---|
| NC_017482.1 | 67892 | G | GA | putative glycosyl hydrolase |
| NC_017482.1 | 91985 | C | CT | |
| NC_017482.1 | 92061 | C | CT | hypothetical protein |
| NC_017482.1 | 133476 | C | CT | |
| NC_017482.1 | 221699 | GT | G | |
| NC_017482.1 | 243226 | GT | G | |
| NC_017482.1 | 267592 | C | CT | myo-inositol catabolismprotein IoID |
| NC_017482.1 | 279665 | GT | G | |
| NC_017482.1 | 287676 | T | TA | conserved hypothetical protein |
| NC_017482.1 | 302968 | CA | C | ADP-ribosylglycohydrolase |
| NC_017482.1 | 303166 | CT | C | ADP-ribosylglycohydrolase |
| NC_017482.1 | 306481 | CT | C | |
| NC_017482.1 | 308789 | A | AGC | 23S ribosomal RNA |
| NC_017482.1 | 308847 | CTT | AAG | 23S ribosomal RNA |
| NC_017482.1 | 308968 | G | CA | 23S ribosomal RNA |
| NC_017482.1 | 308971 | T | C | 23S ribosomal RNA |
| NC_017482.1 | 309008 | T | C | 23S ribosomal RNA |
| NC_017482.1 | 309023 | T | TC | 23S ribosomal RNA |
| NC_017482.1 | 309046 | T | A | 23S ribosomal RNA |
| NC_017482.1 | 309080 | T | C | 23S ribosomal RNA |
| NC_017482.1 | 309093 | G | C | 23S ribosomal RNA |
| NC_017482.1 | 309123 | T | C | 23S ribosomal RNA |
| NC_017482.1 | 309125 | T | C | 23S ribosomal RNA |
| NC_017482.1 | 309215 | G | GA | 23S ribosomal RNA |
| NC_017482.1 | 309736 | T | TC | 23S ribosomal RNA |
| NC_017482.1 | 309748 | G | GC | 23S ribosomal RNA |
| NC_017482.1 | 309761 | A | AT | 23S ribosomal RNA |
| NC_017482.1 | 309781 | G | GT | 23S ribosomal RNA |
| NC_017482.1 | 309783 | T | C | 23S ribosomal RNA |
| NC_017482.1 | 321081 | GA | G | PTS system cellobiose-specific transporter subunit IIC |
| NC_017482.1 | 321381 | GA | G | PTS system cellobiose-specific transporter subunit IIC |
| NC_017482.1 | 323613 | CG | C | phosphopentomutase |
| NC_017482.1 | 326246 | C | CT | |
| NC_017482.1 | 330700 | GA | G | putative DNA helicase |
| NC_017482.1 | 336238 | GA | G | putative transcriptional regulator |
| NC_017482.1 | 336668 | GA | G | putative phosphosugar isomerase |
| NC_017482.1 | 338357 | CA | C | N-acetylglucosamine-6-phosphate deacetylase |
| NC_017482.1 | 341884 | CG | C | putative PTS system IIC component |
| NC_017482.1 | 349070 | CT | C | putative transposase |
| NC_017482.1 | 349854 | GA | G | putative PTS system IIB component |
| NC_017482.1 | 350256 | CT | C | |
| NC_017482.1 | 351500 | TA | T | |
| NC_017482.1 | 365284 | CT | C | |
| NC_017482.1 | 365394 | CA | C | oligopeptide ABC transporter substrate-binding protein |
| NC_017482.1 | 369573 | TG | T | putative multidrug ABC transporter ATP-binding and |
| | | | | permease components |
| NC_017482.1 | 373931 | CT | C | transcriptional regulator |
| NC_017482.1 | 375412 | CT | C | hydroxyethylthiazole kinase |
| NC_017482.1 | 379034 | GA | G | amino acid transporter protein |
| NC_017482.1 | 387272 | TG | T | conserved hypothetical protein |
| NC_017482.1 | 391143 | G | GA | |
| NC_017482.1 | 393952 | G | GT | putative transporter protein |
| NC_017482.1 | 394048 | C | T | putative transporter protein |
| NC_017482.1 | 394056 | T | TC | putative transporter protein |
| NC_017482.1 | 394739 | T | C | putative transporter protein |
| NC_017482.1 | 408930 | GA | G | putative transketolase |
| NC_017482.1 | 410111 | CT | C | |
| NC_017482.1 | 410740 | CT | C | transcriptional regulator |
| NC_017482.1 | 414424 | GA | G | fructose-bisphosphate aldolase |
| NC_017482.1 | 416208 | TA | T | |
| NC_017482.1 | 418411 | CT | C | putative PTS system IICD components |
| NC_017482.1 | 426442 | GA | G | putative amino acid racemase |
| NC_017482.1 | 429366 | CG | C | conserved hypothetical protein |
| NC_017482.1 | 432596 | CG | C | ABC transporter permease |
| NC_017482.1 | 435878 | TA | T | beta-N-acetylglucosaminidase |
| NC_017482.1 | 440712 | GA | G | |
| NC_017482.1 | 441632 | CT | C | |
| NC_017482.1 | 442444 | CT | C | sortase |
| NC_017482.1 | 442597 | CA | C | sortase |
| NC_017482.1 | 444199 | CT | C | cell surface protein SpaB |
| NC_017482.1 | 448849 | T | TA | |
| NC_017482.1 | 453390 | CT | C | conserved hypothetical protein |
| NC_017482.1 | 453574 | TA | T | conserved hypothetical protein |
| NC_017482.1 | 456704 | CA | C | putative transposase |
| NC_017482.1 | 461526 | CG | C | cation-transporting ATPase |
| NC_017482.1 | 463720 | GA | G | putative transposase |
| NC_017482.1 | 469472 | CT | C | |
| NC_017482.1 | 479258 | TG | T | amino acid transporter protein |
| NC_017482.1 | 488627 | CA | C | amidohydrolase |
| NC_017482.1 | 488774 | CT | C | |
| NC_017482.1 | 490334 | GA | G | amino acid ABC transporter ATP-binding protein |
| NC_017482.1 | 495869 | TG | T | lyzozyme |
| NC_017482.1 | 504165 | CT | C | |
| NC_017482.1 | 504357 | GA | G | hypothetical protein |
| NC_017482.1 | 508496 | CT | C | |
| NC_017482.1 | 508626 | GA | G | metal ion ABC transporter substrate binding component |
| NC_017482.1 | 510909 | TG | T | conserved hypothetical protein |
| NC_017482.1 | 511624 | GA | G | conserved hypothetical protein |
| NC_017482.1 | 512012 | CT | C | |
| NC_017482.1 | 514158 | TG | T | transcriptional regulator |
| NC_017482.1 | 515351 | CT | C | dihydroxyacetone kinase |
| NC_017482.1 | 521982 | A | AT | hypothetical protein |
| NC_017482.1 | 523213 | G | GA | hypothetical protein |
| NC_017482.1 | 561033 | C | CT | |
| NC_017482.1 | 565334 | CT | C | transcriptional regulator |
| NC_017482.1 | 627272 | C | T | |
| NC_017482.1 | 639871 | CT | C | putative multidrug ABC transporter ATP-binding component |
| NC_017482.1 | 646360 | GA | G | putative prophage nuclease |
| NC_017482.1 | 648765 | TG | T | ABC transporter ATP-binding protein |
| NC_017482.1 | 651847 | CA | C | |
| NC_017482.1 | 663974 | CT | C | galactokinase |
| NC_017482.1 | 674833 | CG | C | tagatose-6-phosphate kinase |
| NC_017482.1 | 684814 | CT | C | |
| NC_017482.1 | 692073 | CA | C | conserved hypothetical protein |
| NC_017482.1 | 693156 | CA | C | |
| NC_017482.1 | 693804 | C | CAA | |
| NC_017482.1 | 694037 | CT | C | |
| NC_017482.1 | 696764 | CT | C | |
| NC_017482.1 | 696776 | CT | C | hypothetical protein |
| NC_017482.1 | 697163 | CA | C | |
| NC_017482.1 | 702254 | C | T | oxidoreductase |
| NC_017482.1 | 707026 | C | CT | putative glycosyltransferase |
| NC_017482.1 | 735752 | CT | C | |
| NC_017482.1 | 736968 | CT | C | conserved hypothetical protein |
| NC_017482.1 | 742081 | CT | C | |
| NC_017482.1 | 743976 | CT | C | cation-transporting ATPase |
| NC_017482.1 | 745043 | CT | C | cation-transporting ATPase |
| NC_017482.1 | 763402 | GT | G | queuine tRNA-ribosyltransferase |
| NC_017482.1 | 785575 | G | GA | |
| NC_017482.1 | 801800 | C | CT | |
| NC_017482.1 | 811449 | G | T | conserved hypothetical protein |
| NC_017482.1 | 812163 | CT | C | putative phosphoesterase |
| NC_017482.1 | 813534 | GA | G | putative nucleotide phosphoesterase |
| NC_017482.1 | 815059 | G | GT | putative sugar phosphatase |
| NC_017482.1 | 815119 | CA | C | putative sugar phosphatase |
| NC_017482.1 | 818117 | CG | C | thioredoxin reductase |
| NC_017482.1 | 828250 | CT | C | |
| NC_017482.1 | 836793 | GA | G | conserved hypothetical protein |
| NC_017482.1 | 837796 | CT | C | putative phosphoglycerol transferase |
| NC_017482.1 | 840531 | CT | C | |
| NC_017482.1 | 840538 | GA | G | |
| NC_017482.1 | 840838 | A | G | |
| NC_017482.1 | 841522 | A | G | 16S ribosomal RNA |
| NC_017482.1 | 841895 | G | A | 16S ribosomal RNA |
| NC_017482.1 | 842493 | C | CTT | |
| NC_017482.1 | 842510 | G | *** | |
| NC_017482.1 | 842511 | T | TG | |
| NC_017482.1 | 842515 | A | AT | |
| NC_017482.1 | 844170 | G | A | 23S ribosomal RNA |
| NC_017482.1 | 844738 | G | C | 23S ribosomal RNA |
| NC_017482.1 | 866848 | GA | G | conserved hypothetical protein |
| NC_017482.1 | 871190 | CT | C | NAD kinase |
| NC_017482.1 | 871611 | CT | C | pseudouridylate synthase |
| NC_017482.1 | 873165 | CT | C | thiamine biosynthesis lipoprotein |
| NC_017482.1 | 877507 | CT | C | 30S ribosomal protein S14 |
| NC_017482.1 | 879516 | CTT | C | magnesium and cobalt transporter protein |
| NC_017482.1 | 882360 | CT | C | conserved hypothetical protein |
| NC_017482.1 | 895184 | TA | T | conserved hypothetical protein |
| NC_017482.1 | 898415 | CT | C | |
| NC_017482.1 | 901771 | GA | G | protein chain release factor B |
| NC_017482.1 | 904278 | CT | C | |
| NC_017482.1 | 909318 | CT | C | phosphate ABC transporter permease |
| NC_017482.1 | 910057 | CT | C | phosphate ABC transporter permease (different than |
| | | | | previous) |
| NC_017482.1 | 912403 | CT | C | phosphate ABC transporter ATP-binding protein |
| NC_017482.1 | 912696 | GA | G | phosphate uptake regulator |
| NC_017482.1 | 913207 | CT | C | |
| NC_017482.1 | 917329 | GA | G | conserved hypothetical protein |
| NC_017482.1 | 924049 | CT | C | |
| NC_017482.1 | 925010 | CT | C | |
| NC_017482.1 | 932714 | CT | C | conserved hypothetical protein |
| NC_017482.1 | 933859 | CT | C | conserved hypothetical protein |
| NC_017482.1 | 939216 | CT | C | |
| NC_017482.1 | 939713 | T | TA | phosphoglycerate kinase |
| NC_017482.1 | 946072 | CT | C | |
| NC_017482.1 | 946363 | CT | C | esterase |
| NC_017482.1 | 955648 | CT | C | |
| NC_017482.1 | 958526 | CT | C | |
| NC_017482.1 | 961417 | GA | G | ABC transporter permease |
| NC_017482.1 | 967708 | GA | G | |
| NC_017482.1 | 969009 | T | TA | ABC transporter permease |
| NC_017482.1 | 973480 | CG | C | UDP-N-acetylenolpyruvoylglucosamine reductase |
| NC_017482.1 | 975263 | CG | C | Na+/H+ and K+/H+ antiporter |
| NC_017482.1 | 976573 | CA | C | transcriptional regulator |
| NC_017482.1 | 976672 | CT | C | transcriptional regulator |
| NC_017482.1 | 983370 | CT | C | cation transport ATPase |
| NC_017482.1 | 993502 | G | GA | D-fructose-6-phosphate amidotransferase |
| NC_017482.1 | 1003016 | GA | G | DNA methylase |
| NC_017482.1 | 1003057 | CT | C | DNA methylase |
| NC_017482.1 | 1003451 | CT | C | |
| NC_017482.1 | 1005739 | CG | C | 1-acyl-sn-glycerol-3-phosphate acyltransferase |
| NC_017482.1 | 1012031 | T | TAA | |
| NC_017482.1 | 1014034 | GA | G | two-component sensor kinase |
| NC_017482.1 | 1016226 | CT | C | |
| NC_017482.1 | 1019389 | CA | C | nucleotidyltransferase/DNA polymerase |
| NC_017482.1 | 1020536 | GA | G | conserved hypothetical protein |
| NC_017482.1 | 1034559 | TA | T | |
| NC_017482.1 | 1034859 | GA | G | DNA methylase |
| NC_017482.1 | 1038768 | A | G | restriction endonuclease |
| NC_017482.1 | 1039010 | A | G | restriction endonuclease |
| NC_017482.1 | 1039026 | T | C | restriction endonuclease |
| NC_017482.1 | 1039382 | G | A | restriction endonuclease |
| NC_017482.1 | 1039485 | G | T | restriction endonuclease |
| NC_017482.1 | 1039625 | T | C | restriction endonuclease |
| NC_017482.1 | 1039634 | T | C | restriction endonuclease |
| NC_017482.1 | 1039662 | TG | AA | |
| NC_017482.1 | 1039825 | T | A | |
| NC_017482.1 | 1039837 | G | A | |
| NC_017482.1 | 1039840 | G | C | |
| NC_017482.1 | 1039927 | GGT | AAA | |
| NC_017482.1 | 1040029 | C | CT | |
| NC_017482.1 | 1040049 | A | C | |
| NC_017482.1 | 1040051 | C | A | |
| NC_017482.1 | 1047167 | C | CA | |
| NC_017482.1 | 1051823 | G | GA | conserved hypothetical protein |
| NC_017482.1 | 1052476 | G | GA | conserved hypothetical protein (Different than Previous) |
| NC_017482.1 | 1076617 | T | TA | |
| NC_017482.1 | 1102684 | T | TAA | |
| NC_017482.1 | 1105562 | T | TA | |
| NC_017482.1 | 1112190 | G | GA | phage protein |
| NC_017482.1 | 1126408 | G | GA | phage protein (Different than Previous) |
| NC_017482.1 | 1127247 | C | CT | putative phage major structural protein |
| NC_017482.1 | 1141464 | GT | G | conserved hypothetical protein |
| NC_017482.1 | 1211667 | CT | C | oligopeptide ABC transporter substrate-binding protein |
| NC_017482.1 | 1212200 | CT | C | oligopeptide ABC transporter substrate-binding protein |
| NC_017482.1 | 1212851 | GA | G | |
| NC_017482.1 | 1214040 | CT | C | two-component response regulator |
| NC_017482.1 | 1218832 | CT | C | conserved hypothetical protein |
| NC_017482.1 | 1219738 | CT | C | acetyltransferase |
| NC_017482.1 | 1219972 | CT | C | acetyltransferase |
| NC_017482.1 | 1226373 | TG | T | |
| NC_017482.1 | 1226973 | GA | G | conserved hypothetical protein |
| NC_017482.1 | 1232746 | GA | G | |
| NC_017482.1 | 1234549 | CT | C | conserved hypothetical protein |
| NC_017482.1 | 1234657 | CA | C | conserved hypothetical protein (Different than Previous) |
| NC_017482.1 | 1240833 | C | CA | transcription accessory protein |
| NC_017482.1 | 1269918 | T | C | phosphatidylserine/phosphatidylglycerophosphate/ |
| | | | | cardiolipin synthase |
| NC_017482.1 | 1276608 | T | TA | UDP-N-acetylmuramoyl-L-alanyl-D-glutamate synthetase |
| NC_017482.1 | 1309130 | GT | G | putative hydrolase |
| NC_017482.1 | 1333131 | G | GA | putative hydrolase (Different than Previous) |
| NC_017482.1 | 1335127 | C | CT | |
| NC_017482.1 | 1335427 | C | CA | conserved hypothetical protein |
| NC_017482.1 | 1348477 | G | C | putative GTPase |
| NC_017482.1 | 1358927 | CG | C | putative GTPase (Different Than Previous) |
| NC_017482.1 | 1359513 | CT | C | |
| NC_017482.1 | 1361499 | GA | G | |
| NC_017482.1 | 1366986 | CT | C | |
| NC_017482.1 | 1367611 | CA | C | conserved hypothetical protein |
| NC_017482.1 | 1371966 | CA | C | DNA polymerase III subunit alpha |
| NC_017482.1 | 1372807 | CT | C | DNA polymerase III subunit alpha |
| NC_017482.1 | 1377770 | CG | C | conserved hypothetical protein |
| NC_017482.1 | 1379155 | CG | C | integrase |
| NC_017482.1 | 1380446 | CT | C | conserved hypothetical protein |
| NC_017482.1 | 1387773 | CA | C | 30S ribosomal protein S1 |
| NC_017482.1 | 1388528 | CT | C | |
| NC_017482.1 | 1393922 | CA | C | tRNA nucleotidyltransferase/poly(A) polymerase |
| NC_017482.1 | 1394047 | CA | C | tRNA nucleotidyltransferase/poly(A) polymerase |
| NC_017482.1 | 1394523 | CA | C | conserved hypothetical protein |
| NC_017482.1 | 1394557 | C | CA | conserved hypothetical protein |
| NC_017482.1 | 1395559 | CA | C | ABC transporter ATP-binding protein |
| NC_017482.1 | 1396017 | CA | C | ABC transporter ATP-binding protein |
| NC_017482.1 | 1396901 | CA | C | thymidylate synthase |
| NC_017482.1 | 1400211 | CT | C | |
| NC_017482.1 | 1402548 | CA | C | conserved hypothetical protein |
| NC_017482.1 | 1404935 | TG | T | conserved hypothetical protein (Different than Previous) |
| NC_017482.1 | 1413551 | G | GA | ATP-dependent protease peptidase subunit |
| NC_017482.1 | 1413749 | CA | C | ATP-dependent protease peptidase subunit |
| NC_017482.1 | 1415478 | GA | G | |
| NC_017482.1 | 1422044 | CT | C | |
| NC_017482.1 | 1424498 | CA | C | |
| NC_017482.1 | 1432329 | GA | G | antimicrobial peptide ABC transporter ATP-binding |
| | | | | component |
| NC_017482.1 | 1432690 | CA | C | two-component sensor kinase |
| NC_017482.1 | 1433552 | CT | C | two-component response regulator |
| NC_017482.1 | 1437849 | CA | C | fumarylacetoacetate hydrolase |
| NC_017482.1 | 1438278 | A | AT | histidinol-phosphate aminotransferase |
| NC_017482.1 | 1438501 | GA | G | histidinol-phosphate aminotransferase |
| NC_017482.1 | 1438954 | CA | C | histidinol-phosphate aminotransferase |
| NC_017482.1 | 1439491 | CT | C | phosphoribosyl-ATP pyrophosphohydrolase |
| NC_017482.1 | 1439923 | CT | C | phosphoribosyl-AMP cyclohydrolase |
| NC_017482.1 | 1440644 | CT | C | imidazoleglycerol phosphate synthase |
| NC_017482.1 | 1441470 | CT | C | imidazole glycerol phosphate synthase subunit |
| NC_017482.1 | 1442194 | GA | G | imidazoleglycerol-phosphate dehydratase |
| NC_017482.1 | 1443085 | GA | G | histidinol dehydrogenase |
| NC_017482.1 | 1443520 | CA | C | histidinol dehydrogenase |
| NC_017482.1 | 1446024 | GA | G | |
| NC_017482.1 | 1447058 | C | CG | histidinol-phosphate aminotransferase |
| NC_017482.1 | 1447314 | GA | G | histidinol-phosphate aminotransferase |
| NC_017482.1 | 1452112 | GA | G | fibronectin-binding protein |
| NC_017482.1 | 1456685 | CT | C | carbamoylphosphate synthase large subunit |
| NC_017482.1 | 1458356 | GA | G | carbamoylphosphate synthase large subunit |
| NC_017482.1 | 1460775 | GA | G | carbamoylphosphate synthase small subunit |
| NC_017482.1 | 1465416 | CA | C | pseudouridylate synthase |
| NC_017482.1 | 1470351 | CT | C | |
| NC_017482.1 | 1470943 | CT | C | cardiolipin synthetase |
| NC_017482.1 | 1475067 | TA | T | ribonucleotide reductase subunit alpha |
| NC_017482.1 | 1479517 | A | T | endopeptidase |
| NC_017482.1 | 1495933 | CA | C | DNA helicase |
| NC_017482.1 | 1497739 | CT | C | DNA helicase |
| NC_017482.1 | 1506088 | G | GA | mevalonate kinase |
| NC_017482.1 | 1506229 | CT | C | mevalonate kinase |
| NC_017482.1 | 1509707 | CT | C | RNA methyltransferase |
| NC_017482.1 | 1510388 | CA | C | RNA methyltransferase |
| NC_017482.1 | 1514099 | GA | G | putative ABC transporter ATP-binding and permease |
| | | | | components |
| NC_017482.1 | 1516440 | CA | C | putative multidrug ABC transporter ATP-binding and |
| | | | | permease components |
| NC_017482.1 | 1527532 | T | C | |
| NC_017482.1 | 1527637 | T | C | |
| NC_017482.1 | 1527814 | C | A | hypothetical phage protein |
| NC_017482.1 | 1528053 | G | A | hypothetical phage protein |
| NC_017482.1 | 1528058 | A | G | hypothetical phage protein |
| NC_017482.1 | 1528071 | T | C | hypothetical phage protein |
| NC_017482.1 | 1528312 | CC | TT | putative phage lysin |
| NC_017482.1 | 1528352 | G | A | putative phage lysin |
| NC_017482.1 | 1528385 | GG | AA | putative phage lysin |
| NC_017482.1 | 1528837 | C | G | putative phage lysin |
| NC_017482.1 | 1528990 | T | C | putative phage lysin |
| NC_017482.1 | 1528992 | T | C | putative phage lysin |
| NC_017482.1 | 1529099 | T | G | putative phage lysin |
| NC_017482.1 | 1529111 | A | C | putative phage lysin |
| NC_017482.1 | 1529143 | C | A | putative phage lysin |
| NC_017482.1 | 1529194 | G | A | putative phage lysin |
| NC_017482.1 | 1529198 | G | A | putative phage lysin |
| NC_017482.1 | 1529260 | G | A | putative phage lysin |
| NC_017482.1 | 1529290 | G | A | putative phage lysin |
| NC_017482.1 | 1529405 | G | A | putative phage lysin |
| NC_017482.1 | 1529783 | A | C | |
| NC_017482.1 | 1531265 | T | A | phage protein |
| NC_017482.1 | 1531391 | G | T | phage protein |
| NC_017482.1 | 1534866 | GA | G | putative phage tail protein |
| NC_017482.1 | 1537245 | C | CT | putative phage tail protein (Different Than Previous) |
| NC_017482.1 | 1544725 | CA | C | phage portal protein |
| NC_017482.1 | 1547214 | CT | C | phage terminase large subunit |
| NC_017482.1 | 1555391 | T | TA | |
| NC_017482.1 | 1558437 | CA | C | |
| NC_017482.1 | 1560241 | CA | C | acetyltransferase |
| NC_017482.1 | 1561667 | CT | C | putative multidrug ABC transporter ATP-binding component |
| NC_017482.1 | 1562059 | C | CT | |
| NC_017482.1 | 1567743 | CT | C | conserved hypothetical protein |
| NC_017482.1 | 1569332 | CT | C | endonuclease IV |
| NC_017482.1 | 1569353 | CG | C | endonuclease IV |
| NC_017482.1 | 1573603 | CT | C | aspartyl-tRNA synthetase |
| NC_017482.1 | 1574995 | CA | C | histidyl-tRNA synthetase |
| NC_017482.1 | 1582444 | T | TG | GTP pyrophosphokinase |
| NC_017482.1 | 1588651 | GA | G | conserved hypothetical protein |
| NC_017482.1 | 1613998 | CT | C | |
| NC_017482.1 | 1625167 | CA | C | chaperone protein DnaJ |
| NC_017482.1 | 1625650 | C | CA | |
| NC_017482.1 | 1627816 | CT | C | chaperone protein GrpE |
| NC_017482.1 | 1637237 | CT | C | conserved hypothetical protein |
| NC_017482.1 | 1640223 | G | GA | DNA polymerase III subunit alpha |
| NC_017482.1 | 1642735 | CT | C | DNA polymerase III subunit alpha |
| NC_017482.1 | 1664434 | G | A | tRNA (guanine-N1)-methyltransferase |
| NC_017482.1 | 1665131 | T | TA | 16S rRNA processing protein |
| NC_017482.1 | 1676051 | CA | C | |
| NC_017482.1 | 1680240 | T | TA | |
| NC_017482.1 | 1686416 | C | CT | |
| NC_017482.1 | 1706815 | C | CT | conserved hypothetical protein |
| NC_017482.1 | 1712866 | C | CA | |
| NC_017482.1 | 1755907 | CG | C | conserved hypothetical protein |
| NC_017482.1 | 1756285 | GA | G | |
| NC_017482.1 | 1759682 | CT | C | GTP-binding protein |
| NC_017482.1 | 1762550 | CG | C | putative glyoxalase |
| NC_017482.1 | 1775524 | A | AT | conserved hypothetical protein |
| NC_017482.1 | 1780441 | C | CA | dephospho-CoA kinase |
| NC_017482.1 | 1785819 | G | GA | |
| NC_017482.1 | 1787158 | C | CT | putative cell surface protein |
| NC_017482.1 | 1805110 | T | TA | peptidyl-prolyl isomerase |
| NC_017482.1 | 1808818 | G | GA | glycosyltransferase |
| NC_017482.1 | 1818750 | G | GA | hydrolase |
| NC_017482.1 | 1836689 | C | CT | phosphoribosylformylglycinamidine synthase subunit II |
| NC_017482.1 | 1848552 | G | GA | Clp protease ATP-binding subunit |
| NC_017482.1 | 1869561 | C | CT | putative endonuclease |
| NC_017482.1 | 1874602 | C | CA | |
| NC_017482.1 | 1874639 | C | CT | |
| NC_017482.1 | 1880933 | C | T | |
| NC_017482.1 | 1908125 | GT | G | putative melibiose symporter |
| NC_017482.1 | 1909014 | C | CA | putative melibiose symporter |
| NC_017482.1 | 1922176 | C | CG | 23S ribosomal RNA |
| NC_017482.1 | 1922189 | A | AG | 23S ribosomal RNA |
| NC_017482.1 | 1922243 | C | A | 23S ribosomal RNA |
| NC_017482.1 | 1922334 | C | T | 23S ribosomal RNA |
| NC_017482.1 | 1922668 | A | C | 23S ribosomal RNA |
| NC_017482.1 | 1927512 | C | CT | conserved hypothetical protein |
| NC_017482.1 | 1931574 | GT | G | amino acid transporter protein |
| NC_017482.1 | 1935496 | C | CA | cetolactate synthase |
| NC_017482.1 | 2011763 | C | CT | |
| NC_017482.1 | 2022208 | C | CA | |
| NC_017482.1 | 2042887 | G | GA | truncated conserved hypothetical protein |
| NC_017482.1 | 2074838 | GA | G | |
| NC_017482.1 | 2075468 | GA | G | conserved hypothetical protein |
| NC_017482.1 | 2090247 | GA | G | putative glycosyltransferase |
| NC_017482.1 | 2091337 | GT | G | hypothetical protein |
| NC_017482.1 | 2095927 | GT | G | putative glycosyltransferase |
| NC_017482.1 | 2106220 | C | CT | conserved hypothetical protein |
| NC_017482.1 | 2120822 | T | TA | |
| NC_017482.1 | 2153113 | C | T | putative restriction enzyme |
| NC_017482.1 | 2153116 | G | A | putative restriction enzyme |
| NC_017482.1 | 2153125 | C | T | putative restriction enzyme |
| NC_017482.1 | 2159265 | A | G | putative restriction enzyme (Different than Previous) |
| NC_017482.1 | 2159274 | T | C | putative restriction enzyme |
| NC_017482.1 | 2159277 | A | G | putative restriction enzyme |
| NC_017482.1 | 2162030 | CT | C | conserved hypothetical protein |
| NC_017482.1 | 2166054 | CG | C | conserved hypothetical protein (Different than Previous) |
| NC_017482.1 | 2167379 | CT | C | conserved hypothetical protein |
| NC_017482.1 | 2167400 | CT | C | phage protein |
| NC_017482.1 | 2169071 | CT | C | phage protein |
| NC_017482.1 | 2171967 | CG | C | alpha-glucosidase |
| NC_017482.1 | 2175328 | GA | G | sucrose-6-phosphate hydrolase |
| NC_017482.1 | 2178555 | CT | C | cyclopropane-fatty-acyl-phospholipid synthase |
| NC_017482.1 | 2178673 | CT | C | cyclopropane-fatty-acyl-phospholipid synthase |
| NC_017482.1 | 2178929 | CT | C | acetyl-CoA carboxylase alpha subunit |
| NC_017482.1 | 2179547 | CT | C | acetyl-CoA carboxylase alpha subunit |
| NC_017482.1 | 2182116 | CT | C | biotin carboxylase |
| NC_017482.1 | 2187522 | CT | C | 3-oxoacyl-ACP synthase |
| NC_017482.1 | 2202870 | CT | C | beta-lactamase |
| NC_017482.1 | 2203524 | CT | C | beta-lactamase |
| NC_017482.1 | 2204845 | C | CA | |
| NC_017482.1 | 2205399 | CT | C | sortase |
| NC_017482.1 | 2206756 | GA | G | pyruvate oxidase |
| NC_017482.1 | 2208169 | TA | T | |
| NC_017482.1 | 2211250 | CA | C | glycine/betaine/carnitine ABC transporter ATP-binding |
| | | | | component |
| NC_017482.1 | 2216928 | CT | C | threonine synthase |
| NC_017482.1 | 2219866 | CT | C | |
| NC_017482.1 | 2221370 | G | GA | conserved hypothetical protein |
| NC_017482.1 | 2225826 | GA | G | aromatic amino acid aminotransferase |
| NC_017482.1 | 2258229 | C | CT | |
| NC_017482.1 | 2286291 | C | CT | |
| NC_017482.1 | 2317045 | G | GA | conserved hypothetical protein |
| NC_017482.1 | 2324421 | CA | C | DNA polymerase III subunits gamma and tau |
| NC_017482.1 | 2326394 | GA | G | putative cytosine/adenosine deaminase |
| NC_017482.1 | 2326777 | CT | C | putative cytosine/adenosine deaminase |
| NC_017482.1 | 2327510 | CT | C | 16S rRNA methylase |
| NC_017482.1 | 2330723 | TA | T | conserved hypothetical protein |
| NC_017482.1 | 2330744 | GA | G | conserved hypothetical protein |
| NC_017482.1 | 2330831 | CT | C | |
| NC_017482.1 | 2344299 | CT | C | |
| NC_017482.1 | 2352730 | GA | G | putative glycosyltransferase |
| NC_017482.1 | 2352944 | GA | G | putative glycosyltransferase |
| NC_017482.1 | 2353082 | CA | C | putative glycosyltransferase |
| NC_017482.1 | 2353890 | CA | C | putative glycosyltransferase (Different than previous) |
| NC_017482.1 | 2359603 | CA | C | magnesium-translocating P-type ATPase |
| NC_017482.1 | 2362781 | CT | C | amidase |
| NC_017482.1 | 2362858 | CT | C | amidase |
| NC_017482.1 | 2368535 | CA | C | nucleoside 2-deoxyribosyltransferase |
| NC_017482.1 | 2368888 | TA | T | conserved hypothetical protein |
| NC_017482.1 | 2372794 | T | TA | 1,4-dihydroxy-2-naphthoate octaprenyltransferase |
| NC_017482.1 | 2373836 | CT | C | thiamin biosynthesis lipoprotein |
| NC_017482.1 | 2376365 | C | CT | |
| NC_017482.1 | 2384202 | GA | G | conserved hypothetical protein |
| NC_017482.1 | 2384548 | CT | C | |
| NC_017482.1 | 2384989 | CT | C | conserved hypothetical protein |
| NC_017482.1 | 2386685 | CT | C | putative multidrug ABC transporter ATP-binding component |
| NC_017482.1 | 2391726 | CT | C | |
| NC_017482.1 | 2396357 | CA | C | Hypothetical Protein |
| NC_017482.1 | 2402485 | CT | C | conserved hypothetical protein |
| NC_017482.1 | 2404129 | CT | C | |
| NC_017482.1 | 2404160 | CT | C | |
| NC_017482.1 | 2404392 | CT | C | putative multidrug ABC transporter ATP-binding and |
| | | | | permease components |
| NC_017482.1 | 2405860 | GA | G | |
| NC_017482.1 | 2411332 | CT | C | dUTPase |
| NC_017482.1 | 2421632 | CT | C | pyruvate phosphate dikinase |
| NC_017482.1 | 2422557 | GA | G | pyruvate phosphate dikinase |
| NC_017482.1 | 2424354 | GA | G | |
| NC_017482.1 | 2426043 | C | CT | |
| NC_017482.1 | 2427696 | C | CA | |
| NC_017482.1 | 2431104 | CT | C | conserved hypothetical protein |
| NC_017482.1 | 2432738 | C | CT | |
| NC_017482.1 | 2433948 | CA | C | |
| NC_017482.1 | 2438362 | CG | C | sortase |
| NC_017482.1 | 2438982 | CT | C | sortase |
| NC_017482.1 | 2440534 | CT | C | putative cell surface protein SpaD |
| NC_017482.1 | 2441315 | GA | G | putative cell surface protein SpaE |
| NC_017482.1 | 2443799 | GA | G | putative cell surface protein SpaF |
| NC_017482.1 | 2447222 | TA | T | |
| NC_017482.1 | 2449295 | CT | C | |
| NC_017482.1 | 2454539 | CA | C | |
| NC_017482.1 | 2459727 | CA | C | putative two-component sensor kinase |
| NC_017482.1 | 2462550 | CT | C | |
| NC_017482.1 | 2465207 | CT | C | |
| NC_017482.1 | 2466135 | CT | C | |
| NC_017482.1 | 2467784 | CT | C | putative transcriptional regulator |
| NC_017482.1 | 2471605 | CT | C | putative oxidoreductase |
| NC_017482.1 | 2474382 | CA | C | conserved hypothetical protein |
| NC_017482.1 | 2474594 | CT | C | fumarate hydratase |
| NC_017482.1 | 2475687 | CT | C | fumarate hydratase |
| NC_017482.1 | 2476120 | CG | C | conserved hypothetical protein |
| NC_017482.1 | 2476545 | CT | C | conserved hypothetical protein |
| NC_017482.1 | 2477119 | C | CA | conserved hypothetical protein (Different than Previous) |
| NC_017482.1 | 2481736 | GA | G | cation transport ATPase |
| NC_017482.1 | 2481784 | CA | C | cation transport ATPase |
| NC_017482.1 | 2486376 | CA | C | conserved hypothetical protein |
| NC_017482.1 | 2489781 | CT | C | manganese ABC transporter ATP-binding component |
| NC_017482.1 | 2491455 | GA | G | metal ion ABC transporter substrate binding component |
| NC_017482.1 | 2491575 | CA | C | metal ion ABC transporter substrate binding component |
| NC_017482.1 | 2492208 | CT | C | |
| NC_017482.1 | 2493360 | CT | C | conserved hypothetical protein |
| NC_017482.1 | 2494764 | GA | G | metal ion ABC transporter substrate binding component |
| NC_017482.1 | 2494808 | GA | G | metal ion ABC transporter substrate binding component |
| NC_017482.1 | 2500081 | T | TA | |
| NC_017482.1 | 2508452 | T | TA | |
| NC_017482.1 | 2510919 | C | CT | putative multidrug ABC transporter ATP-binding and |
| | | | | permease components |
| NC_017482.1 | 2514037 | CT | C | putative ABC transporter permease |
| NC_017482.1 | 2540919 | T | TC | |
| NC_017482.1 | 2541585 | C | CA | |
| NC_017482.1 | 2584287 | GA | G | conserved hypothetical protein |
| NC_017482.1 | 2584654 | TC | T | transcriptional regulator |
| NC_017482.1 | 2595078 | GA | G | glutamate synthase large subunit |
| NC_017482.1 | 2597285 | CA | C | conserved hypothetical protein |
| NC_017482.1 | 2598939 | C | CT | |
| NC_017482.1 | 2605539 | CT | C | |
| NC_017482.1 | 2605721 | CT | C | |
| NC_017482.1 | 2611757 | CA | C | conserved hypothetical protein |
| NC_017482.1 | 2613080 | GA | G | |
| NC_017482.1 | 2614951 | CT | C | lipoate-protein ligase |
| NC_017482.1 | 2616766 | C | CT | phosphohydrolase |
| NC_017482.1 | 2617149 | CT | C | hydrolase |
| NC_017482.1 | 2621383 | CT | C | UDP-N-acetylglucosamine pyrophosphorylase |
| NC_017482.1 | 2623503 | TA | T | conserved hypothetical protein |
| NC_017482.1 | 2626663 | T | TA | |
| NC_017482.1 | 2631116 | CA | C | DNase |
| NC_017482.1 | 2634966 | CA | C | two-component response regulator |
| NC_017482.1 | 2661444 | T | TC | |
| NC_017482.1 | 2700105 | C | CA | |
| NC_017482.1 | 2757467 | AT | A | putative transposase |
| NC_017482.1 | 2780032 | T | TA | |
| NC_017482.1 | 2813504 | A | AT | PTS system galactitol-specific IIC component |
| NC_017482.1 | 2850347 | C | CT | |
| NC_017482.1 | 2860412 | GA | G | oligopeptide ABC transporter substrate-binding protein |
| NC_017482.1 | 2860492 | CT | C | oligopeptide ABC transporter substrate-binding protein |
| NC_017482.1 | 2862035 | CA | C | conserved hypothetical protein |
| NC_017482.1 | 2862455 | CA | C | conserved hypothetical protein |
| NC_017482.1 | 2864292 | GA | G | cation transport ATPase |
| NC_017482.1 | 2865651 | G | GA | |
| NC_017482.1 | 2870662 | CT | C | conserved hypothetical protein |
| NC_017482.1 | 2874336 | CT | C | two-component sensor kinase |
| NC_017482.1 | 2881415 | CA | C | conserved hypothetical protein |
| NC_017482.1 | 2886599 | CT | C | pyrophosphatase |
| NC_017482.1 | 2898266 | CA | C | transcriptional regulator |
| NC_017482.1 | 2900593 | CT | C | conserved hypothetical protein |
| NC_017482.1 | 2903157 | CA | C | conserved hypothetical protein |
| NC_017482.1 | 2903600 | GA | G | conserved hypothetical protein (Different than Previous) |
| NC_017482.1 | 2904564 | GA | G | conserved hypothetical protein |
| NC_017482.1 | 2913910 | CT | C | conserved hypothetical protein (Different than Previous) |
| NC_017482.1 | 2916035 | CA | C | putative phosphotransferase |
| NC_017482.1 | 2921341 | CA | C | |
| NC_017482.1 | 2922658 | CT | C | methanol dehydrogenase |
| NC_017482.1 | 2923668 | CA | C | conserved hypothetical protein |
| NC_017482.1 | 2926304 | GA | G | putative protease |
| NC_017482.1 | 2926927 | CA | C | |

***GCTCTCTTTGGTTGTTGGGGGGCCCCCTTTTTAAAGGGGGGGGGTGGTGTTTAATTCAT
TTTTACCCCTCAAAATTTCTTTTTTTTTAAAAAACCTTTTGGGGGATTAATTCACTTGGGAGAGCCCCT
CCTTTTCAAGCAGGAGGTCATCGGTTCGATCCCGTTATCCTCCATTGGCCGTCAGGCCGATCGTTGA
CGTTAGT

TABLE 2

| | # | |
|---|---|---|
| | changes | |
| | in | |
| | single | Range of Bases |
| Gene | gene | in Genome |
| ADP-ribosylglycohydrolase BAI40821.1 | 2 | 302660-303631 |
| 23S ribosomal RNA LRHM_23SrRNA01 | 17 | 308563-311482 |
| PTS system cellobiose-specific | 2 | 320173-321489 |
| transporter subunit IIC BAI40834.1 | | |
| putative transporter protein BAI40899.1 | 4 | 393415-394803 |
| sortase BAI40952.1 | 2 | 441889-442978 |
| conserved hypothetical protein | 2 | 452680-454770 |
| BAI40961.1 | | |
| conserved hypothetical protein | 2 | 510819-511784 |
| BAI41012.1 | | |
| cation-transporting ATPase BAI41240.1 | 2 | 743184-745532 |
| putative sugar phosphatase BAI41309.1 | 2 | 814949-815725 |
| 16S ribosomal RNA LRHM_16SrRNA03 | 2 | 840850-842423 |
| 23S ribosomal RNA 2 | 2 | 842641-845560 |
| LRHM_23SrRNA03 | | |
| transcriptional regulator BAI41452.1 | 2 | 976535-977074 |
| restriction endonuclease BAI41509.1 | 7 | 1036681-1039656 |
| DNA methylase BAI41477.1 | 2 | 1002221-1003234 |
| oligopeptide ABC transporter substrate- | 2 | 1211139-1212779 |
| binding protein BAI41697.1 | | |
| acetyltransferase BAI41706.1 | 2 | 1219416-1220279 |
| DNA polymerase III subunit alpha | 2 | 1370585-1373881 |
| BAI41844.1 | | |
| tRNA nucleotidyltransferase/poly(A) | 2 | 1392900-1394096 |
| polymerase BAI41865.1 | | |
| ABC transporter ATP-binding protein | 2 | 1394825-1396738 |
| BAI41867.1 | | |
| ATP-dependent protease peptidase | 2 | 1414203-1415612 |
| subunit BAI41887.1 | | |
| histidinol-phosphate aminotransferase | 3 | 1438106-1439212 |
| BAI41906.1 | | |
| histidinol dehydrogenase BAI41913.1 | 2 | 1442903-1444183 |
| histidinol-phosphate aminotransferase | 2 | 1446476-1447573 |
| BAI41916.1 | | |
| DNA helicase BAI41962.1 | 2 | 1495565-1498366 |
| mevalonate kinase BAI41965.1 | 2 | 1506071-1507006 |
| RNA methyltransferase BAI41968.1 | 2 | 1509125-1510456 |
| hypothetical phage protein BAI41983.1 | 4 | 1527712-1528098 |
| putative phage lysin BAI41984.1 | 14 | 1529289-1529443 |
| phage protein BAI41989.1 | 2 | 1530506-1533439 |
| endonuclease IV BAI42022.1 | 2 | 1569245-1570132 |
| DNA polymerase III subunit alpha | 2 | 1639271-1643605 |
| BAI42081.1 | | |
| putative melibiose symporter BAI42331.1 | 2 | 1907604-1909037 |
| 23S ribosomal RNA LRHM_23SrRNA04 | 5 | 1920458-1923378 |
| putative restriction enzyme BAI42541.1 | 3 | 2153042-2155261 |
| putative restriction enzyme BAI42543.1 | 3 | 2156376-2159933 |
| conserved hypothetical protein | 2 | 2165105-2166289 |
| BAI42547.1 | | |
| phage protein BAI42548.1 | 2 | 2166639-2169371 |
| cyclopropane-fatty-acyl-phospholipid | 2 | 2177506-2178687 |
| synthase BAI42555.1 | | |
| beta-lactamase BAI42585.1 | 2 | 2202634-2203731 |
| putative cytosine/adenosine deaminase | 2 | 2326328-2326840 |
| BAI42706.1 | | |
| conserved hypothetical protein | 2 | 2328150- 2330759 |
| BAI42709.1 | | |
| putative glycosyltransferase BAI42722.1 | 3 | 2352077-2353573 |
| amidase BAI42730.1 | 2 | 2362600-2364051 |
| pyruvate phosphate dikinase BAI42788.1 | 2 | 2421615-2424245 |
| sortase BAI42805.1 | 2 | 2438170-2438994 |
| fumarate hydratase BAI42845.1 | 2 | 2474467-2475846 |
| conserved hypothetical protein | 2 | 2861862-2863055 |
| BAI43223.1 | | |
| metal ion ABC transporter substrate | 2 | 2491123-2492040 |
| binding component BAI42862.1 | | |
| metal ion ABC transporter substrate | 2 | 2493938-2494840 |
| binding component BAI42865.1 | | |
| cation transport ATPase BAI42850.1 | 2 | 2479323-2482040 |
| oligopeptide ABC transporter substrate- | 2 | 2859325-2860935 |
| binding protein BAI43222.1 | | |
| conserved hypothetical protein | 2 | 2476021-2476764 |
| BAI42846.1 | | |
| conserved hypothetical protein | 2 | 1394221-1394676 |
| BAI41866.1 | | |
| carbamoylphosphate synthase large | 2 | 1456547-1459729 |
| subunit BAI41926.1 | | |
| conserved hypothetical protein | 2 | 2900568-2900912 |
| BAI43260.1 | | |
| conserved hypothetical protein | 2 | 2903494-2905191 |
| BAI43264.1 | | |

In certain embodiments, the genetic alteration is deletion of at least one base pair. In certain embodiments, the genetic alteration is the addition of at least one base pair. In certain embodiments, the genetic alteration increases gut persistence by at least 1%-500%. In preferred embodiments, the alteration increases gut persistence by at least 100%-300%. In certain embodiments, the genetic alteration increases gut persistence by approximately 300%.

The term “genetic alteration” as used herein refers to a change from the wild-type or reference sequence of one or more nucleic acid molecules. Genetic alterations include without limitation, base pair substitutions, additions and deletions of at least one nucleotide from a nucleic acid molecule of known sequence.

A “persistence-associated genetic alteration” is a genetic alteration which is associated with an altered gut persistence not observed in microorganisms lacking this genetic alteration. The presence of such alterations in a microorganism convey an increased gut persistence phenotype to the microorganisms. These increase gut persistence phenotypes include without limitation, gut persistence, decreased elimination rate, increased bile salt resistance, or altered binding to Caco-2 cells when compared to microorganism lacking said alteration. Such markers may include but are not limited to nucleic acids, proteins encoded thereby, or other small molecules. Thus, the phrase “persistence-associated SNP containing nucleic acid” is encompassed by the above description.

A “single nucleotide polymorphism (SNP)” refers to a change in which a single base in the DNA differs from the usual base at that position. These single base changes are called SNPs or “snips.” Millions of SNP's have been cataloged in bacterial genome. Some SNPs are markers of disease, other SNPs are responsible for disease. Other SNPs are normal variations in the genome.

“Target nucleic acid” as used herein refers to a previously defined region of a nucleic acid present in a complex nucleic acid mixture wherein the defined wild-type region contains at least one known nucleotide variation which may or may not be associated with gut-persistence but is informative of an altered response to gut stressor conditions. The nucleic acid molecule may be isolated from a natural source by cDNA cloning or subtractive hybridization or synthesized manually. The nucleic acid molecule may be synthesized manually by the triester synthetic method or by using an automated DNA synthesizer. When cloning a target nucleic acid comprising a deletion, the skilled artisan is well aware of methods for selecting nucleic acids of a sufficient length flanking the affected region to facilitate cloning the region into a vector of choice.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

By the use of the term “enriched” in reference to nucleic acid it is meant that the specific DNA or RNA sequence constitutes a significantly higher fraction (2-5 fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10-6-fold purification of the native message. Thus, purification of at least one order of magnitude, preferably two or three orders, and more preferably four or five orders of magnitude is expressly contemplated. Thus, the term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus, if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence which hybridizes to any genetic alteration but does not hybridize to other nucleotides. Also, polynucleotide which “specifically hybridizes” may hybridize only to a genetic alteration shown in the Tables contained herein. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989):

Tₘ=81.5° C.+16.6 Log [Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regard to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 g/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “oligonucleotide,” as used herein is defined as a nucleic acid molecule comprised of two or more ribo- or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides are at least about 10 nucleotides in length, more preferably at least 15 nucleotides in length, more preferably at least about 20 nucleotides in length.

In certain embodiments, the persistence genetic alteration is introduced to the microorganism using a vector.

### Vectors and Transgenes

The microorganisms disclosed herein can be transformed to express nucleic acids harboring sequences of interest for a variety of purposes. Transformation refers to the stable integration of transforming DNA and constitutive expression into the target genome that is transmitted to the microorganism and its progeny. In certain embodiments, the microorganism is transformed to comprise at least one of the genetic modifications described above.

In certain embodiments, the microorganism is transformed to express a transgene. The term “transgene” refers to a non-marker sequence encoding a product which is useful in biology and medicine, such as proteins, peptides, RNA, enzymes, dominant negative mutants, or catalytic RNAs. Desirable RNA molecules include tRNA, dsRNA, ribosomal RNA, catalytic RNAs, siRNA, small hairpin RNA, trans-splicing RNA, and antisense RNAs. Examples of useful transgenes include, without limitation, GLP1 (glucagon-like peptide-1), sTNFR1 (TNF receptor antagonist), IL-1RA (Interleukin-1 receptor antagonist), sGP130, EPO (erythropoietin), PTH (parathyroid hormone), insulin, or enfuvirtide and derivatives thereof.

In certain embodiments, the invention provides methods comprising delivering one or more polynucleotides, such as or one or more vectors as described herein, one or more transcripts thereof, and/or one or proteins transcribed therefrom, to a host cell. In some aspects, the invention further provides cells produced by such methods, and organisms (such as bacteria, animals, plants, or fungi) comprising or produced from such cells. In some embodiments, a CRISPR enzyme in combination with (and optionally complexed with) a gRNA is delivered to a cell. Conventional viral and non-viral based gene transfer methods can be used to introduce nucleic acids in mammalian cells or target tissues. Such methods can be used to administer nucleic acids encoding components of a CRISPR system to cells in culture, or in a host organism.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, PEG-fusion, a viral vector, a naked plasmid and the like.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6 (10): 1149-1154 (1988); Vigne, Restorative Neurology and Neuroscience 8:35-36 (1995); Kremer & Perricaudet, British Medical Bulletin 51 (1): 31-44 (1995); Haddada et al., in Current Topics in Microbiology and Immunology Doerfler and Bihm (eds) (1995); and Yu et al., Gene Therapy 1:13-26 (1994). Methods of non-viral delivery of nucleic acids include lipofection, nucleofection, microinjection, biolistics, virosomes, liposomes, immunoliposomes, polycation or lipid-nucleic acid conjugates, lipid nanoparticles, artificial virions, virus-like particles, naked DNA, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g., in vitro or ex vivo administration) or target tissues (e.g., in vivo administration).

The preparation of lipid nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787). Other lipid nanoparticle formulations are disclosed in U.S. Pat. Nos. 11,066,355; 11,059,807; US patent publications 2021/0106538 and 2021/0113466.

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue.

Retroviral vectors comprise cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), simian immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

Packaging cells are typically used to form virus particles that are capable of infecting a host cell. Such cells include HEK 293 cells, which package adenovirus, and y2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line.

For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. In one aspect, the invention provides for methods of modifying a target polynucleotide in a eukaryotic cell, which may be in vivo, ex vivo or in vitro. In some embodiments, the method comprises sampling a cell or population of cells from a human or non-human animal, and modifying the cell or cells. Culturing may occur at any stage ex vivo. The cell or cells may be re-introduced into the human or non-human animal.

In one aspect, the invention provides for methods of modifying a target polynucleotide in a eukaryotic cell. In some embodiments, the method comprises allowing an adenine base editor (ABE) CRISPR complex to bind to the target polynucleotide to effect correction of a mutation in said target polynucleotide thereby modifying the target polynucleotide, wherein the CRISPR complex comprises the ABE CRISPR enzyme complexed with a gRNA hybridized to a target sequence within said target polynucleotide.

### Methods and Uses for Treating Inflammatory Diseases

The probiotic formulations of the present disclosure can be administered to subjects in need thereof including humans and non-human animals, for example, cows, pigs, poultry and fish. Administration to humans includes administration by a medical professional and self-administration. In general, in order to achieve a health benefit, multiple doses of the probiotic formulations are administered, for example daily for a period of at least one week, at least two weeks, at least three weeks, at least six weeks, at least nine weeks, or at least twelve weeks. In one embodiment, the probiotics can be administered for the remaining duration of a subject's life.

In certain embodiments, the probiotic formulations are administered in a method for treating an inflammatory bowel disease.

The term “inflammatory bowel disease” refers to a disorder or disease characterized by inflammatory activity (inflammation) in the gastrointestinal tract, particularly inflammatory conditions of the large intestine and/or small intestine. Inflammatory bowel diseases are often chronic conditions of uncertain etiology, characterized by recurrent episodes of abdominal pain, often with diarrhea. Examples of inflammatory bowel diseases include, without limitation: Crohn's disease (also referred to as regional enteritis, terminal ileitis, or granulomatous ileocolitis), colitis (e.g., ulcerative colitis, indeterminate colitis, lymphocytic colitis, ischaemic colitis, diversion colitis, microscopic colitis, infective colitis, or collagenous colitis), Bahcet's syndrome, idiopathic inflammation of the small and/or proximal intestine, and IBD-related diarrhea. In a particular embodiment, the “inflammatory bowel disease” is ulcerative colitis (UC) or Crohn's disease (CD). The methods of the instant invention may further comprise the administration of at least one other therapeutic for the inflammatory bowel disease being treated.

The term “treatment,” as used herein, includes any administration or application of a therapeutic for a disease or disorder in a subject, and includes inhibiting the disease, arresting its development, relieving the symptoms of the disease, or preventing occurrence or reoccurrence of the disease or symptoms of the disease.

The total treatment dose or doses (when two or more targets are to be modulated) can be administered to a subject as a single dose or can be administered using a fractionated treatment protocol, in which multiple/separate doses are administered over a more prolonged period of time, for example, over the period of a day to allow administration of a daily dosage or over a longer period of time to administer a dose over a desired period of time. One skilled in the art would know that the amount of therapeutic agent required to obtain an effective dose in a subject depends on many factors, including the age, weight and general health of the subject, as well as the route of administration and the number of treatments to be administered. In view of these factors, the skilled artisan would adjust the particular dose so as to obtain an effective dose for treating an individual having the inflammatory disease.

The effective dose of the probiotic formulations will depend on the mode of administration, and the weight of the individual being treated. The dosages described herein are generally those for an average adult but can be adjusted for the treatment of children. The dose will generally range from about 0.001 mg to about 1000 mg.

In an individual suffering from a more severe form of the disease, administration of therapeutic agents can be particularly useful when administered in combination, for example, with a conventional agent for treating such a disease. The skilled artisan would administer therapeutic agent(s), alone or in combination and would monitor the effectiveness of such treatment using routine methods such as neurological or pulmonary function determination, radiologic or immunologic assays, or, where indicated, histopathologic methods.

Administration of the pharmaceutical preparation is preferably in an “effective amount” this being sufficient to show benefit to the individual. This amount prevents, alleviates, abates, or otherwise reduces the severity of the inflammatory disease symptoms in a patient. Treatment of patients having the inflammatory disease with an efficacious amount of the probiotic formulations may produce improvements in neurological function, respiratory function, tapering of concomitant medication usage, or increased survival.

The pharmaceutical preparation is formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

Pharmaceutical compositions that are useful in the methods of the invention may be administered systemically in oral solid and liquid formulations or other known routes of administration. In addition to the agent(s) useful for treating an inflammatory disease, the pharmaceutical compositions may contain pharmaceutically-acceptable carriers and other ingredients known to enhance and facilitate drug administration. Thus, such compositions may optionally contain other components, such as adjuvants, e.g., aqueous suspensions of aluminum and magnesium hydroxides, and/or other pharmaceutically acceptable carriers, such as saline. Other possible formulations, such as nanoparticles, liposomes, resealed erythrocytes, and immunologically based systems may also be used to deliver/administer the appropriate agent to a patient according to the methods of the invention. The use of nanoparticles to deliver such agents, as well as cell membrane permeable peptide carriers that can be used are described in Crombez et al., Biochemical Society Transactions v35: p44 (2007).

The pharmaceutical compositions can also comprise anti-inflammatory agents for co-administration to further alleviate symptoms of the disease. These include, without limitation, corticosteroids, aspirin, celecoxib, diclofenac, diflunisal, etodolac, ibuprofen, indomethacin, ketoprofen, ketorolac, nabumetone, naproxen, oxaprozin, piroxicam, salsalate, sulindac, tolmetin, interleukin (IL)-1 receptor antagonist, IL-4, IL-6, IL-10, IL-11, IL-13, cytokine receptors for IL-1, tumor necrosis factor-alpha, IL-18 and derivatives and biosimilars thereof. Examples of inflammatory bowel disease therapeutics include, without limitation: anti-inflammatory agents, TNF alpha inhibitors (e.g., monoclonal antibodies (e.g., infliximab, adalimumab, certolizumab, and golimumab), receptor fusion proteins (e.g., etanercept)), steroids, aminosalicylates (e.g., balsalazide, mesalamine, olsalazine, sulfasalazine), and immunomodulators or immunosuppressants (e.g., azathioprine, micronutrients (e.g., zinc, berberine)).

The following materials and methods are provided to facilitate the practice of the present invention.

Culture of Lactobacilli rhamnosus

Lactobacillus rhamnosus (ATCC 53103) was grown in RPI brand MRS Broth which was prepared as per the manufacturer guidelines. The MRS broth was autoclaved before use to ensure no contamination. The Lactobacillus was grown at 37° C. with 5% CO₂ for 18 hours. Evolutions were stored at −80° C. with 10% glycerol for long term storage.

Isolation and Ex Vivo Culture of Lactobacillus rhamnosus after Gavage

The Lactobacillus rhamnosus was administered to two individually housed mice by oral gavage at a concentration of roughly 0.5×10¹⁰ Colony Forming Units (CFU) mL⁻¹. The bacteria was pelleted and administered in 0.5 mL of water via 2 mL syringe. Lactobacillus rhamnosus was found to have a natural resistance to vancomycin, which was used for selection [9]. When the supernatant of the mouse fecal samples were plated prior to gavage, no vancomycin resistant colonies were detected thus demonstrating the antibiotic-based plating method was fully selective.

The fecal matter was collected every day from the cages, and five to six pellets were rehydrated with 1 mL of deionized water. After 10 minutes the samples were vortexed until the solution appeared homogenous. The samples were centrifuged at 2000 rpm for 2 minutes, and 100 μL of the supernatant was collected and plated onto vancomycin plates at dilutions of 1:10, 1:100 and 1:1000 and grown 37° C. in anaerobic chamber created using AnaeroPack. This was done until the supernatant CFU total was less than 10. Then colonies were selected from the plates and then grown again in MRS broth and refed to the mice repeating the same process twice. Samples were additionally collected until the CFU reached zero.

### Next Generation DNA Sequencing

Evolution 1 was grown in MRS broth and the cells pelleted and washed twice. The DNA was extracted from the pelleted isolate using the invitrogen charges with gDNA kit and used for nanopore sequencing as follows. The DNA was fragmented via sonication to obtain DNA approximately 8 Kb in size. It was then barcoded using the kit SQK-LSK 109 with the PCR barcoding expansion 1-12 EXP-PBC0001 supplied by Oxford Nanopore. The barcoding was performed following the respective protocols for DNA sample preparation for sequencing. Next a R 9.4.1 flow cell was primed according to the flow cell priming kit (EXP-FLP002) protocol and 50 fmol loaded after mixing it with the nanopore loading beads and sequencing buffer supplied with the kits. The Sequencing was run in the MinION Mk1C and set it for real-time data acquisition and base calling.

### Sequencing Data Analysis

The MinKNOW app built-in the MinION Mk1C recognized and segregated the fastq sequences pertaining to the isolate according to the barcode which was used (barcode 4). The fastq file was concatenated using FastCat and the concatenated file was used to create a draft assembly using FLYE. The draft assembly was then polished through Medaka. The resulting final consensus assembly was compared to the ORFeome reference file for Lactobacillus rhamnosus GG (ATCC 53103) found in NCBI (RefSeq: NC_017482.1) uploaded locally to identify variants using the Medaka haploid option. The filtering was performed with Samtools and R software.

### Bile Salt Resistance Testing

MRS broth was supplemented with Millipore Brand Bile salts at the following concentrations: 0.5%, 0.75%, and 1.0%. Corning 96 well plates were filled with 200 μL of broth and inoculated with 2 μl of culture of each wild type, evolution 1 and evolution 2 and grown at 37° C. in a Varioskan Lux Microplate Reader.

Three different trials were run in which absorbance readings were taken at a wavelength of 600 nm, with incremental measurements taken using a kinetic loop. Absorbance readings were collected every hour for 15 hours.

### Culture of Caco-2 Cells

Caco-2 Cells were grown in RPMI supplemented with 10% FBS at 37° C. in 75 mm flasks before being transferred to Corning 48 well plates. They were passaged as instructed by the manufacturer once cells reached 80% confluency. Cells were trypsinized using TripleE trypsin. Within the Corning 48 well plates the cells were purposely over-seeded to speed up the process of growing a complete monolayer with a starting value of 55,000 cells per well. Media was added to the wells to a total volume of 750 μL per well. The cells were grown until the wells reached over 90% confluency, indicating a clear monolayer. Media was replaced every two days.

In Vitro Perfusion Assay for Adhesion Testing of Lactobacillus rhammosus on Caco-2 Cells

Lactobacilli were perfused through a multi-stream perfusion setup as previously described by Erickson et. al. [10]. The New Era Model Syringe Pump was set a rate of 0.3 mL/h while the Biorad Fraction collector was set to have fractions be collected every two minutes. All tubing used for the system was autoclaved prior to the perfusion. Three, sterile 20 mL plastic syringes were each filled with 15 mL of a liquid Lactobacilli culture, one for the wild type and one for each of the two evolutions. The perfusion was run until the entire liquid culture of each syringe was completely perfused through the system.

Once the perfusion was completed, the procedure completed was based on that performed by Reddy and Austin [11]. The well plates were then washed with 200 μL of PBS 5 separate times to remove non-adherent bacteria. Afterwards, 500 μL of MRS broth was added to each well and the wells were scraped for 30 seconds using a cell scrapper. The broth was then transferred out of the well and diluted to concentrations of 1:100 and 1:1000. Then 100 μL was plated onto MRS agar plates containing vancomycin at a concentration of 10 μL mL⁻¹. The plates were then grown overnight at 37° C. in an anaerobic chamber created using AnaeroPack.

### Heterologous Gavages of Evolutionary Isolates

Wild-type Lactobacillus rhamnosus, along with the two evolutionary isolates were administered to a fresh, individually housed mouse by gavage at a concentration of roughly 0.5×10¹⁰ Colony Forming Units (CFU) mL⁻¹. As with the autologous gavages, the fecal matter was collected every day from the cages, and five to six pellets were rehydrated with 1 mL of deionized water. After 10 minutes the samples were vortexed until the solution appeared homogenous. The samples were centrifuged at 2000 rpm for 2 minutes, and 100 μL of the supernatant was collected and plated onto vancomycin plates at dilutions of 1:10, 1:100 and 1:1000. The samples were grown at 37° C. in an anaerobic chamber created using AnaeroPack. Samples were collected until the CFU reached zero.

### Equations

To design an equation for the total drug accumulation, a one-compartment, multi-dose IV-bolus model for concentration was used, as it is the simplest model to show the accumulation of bacteria in the gut with daily ingestion. Assumptions that were made to simplify the model include: the bacteria produce ‘Drug X’ at a constant rate, the bacteria produce ‘Drug X’ as soon as they enter the GI Tract and continue until they leave, and that every day the same amount of the initial inoculum survives passage into the intestinal tract. The equation for a multi-dose IV bolus model concentration after t time is as follows:

$$C ( t ) = \frac{D}{V} \frac{{e}^{- ( n * ke * 2.303 * T )}}{{e}^{- ( ke * 2.303 * T )}} * {e}^{ke * 2.303 * t}$$

$${}^{*} units = CFU {mL}^{- 1}$$

A table of values defining each part of the equation can be found in Table 3. The equation was converted to drug concentration by multiplying it by the secretion rate found from in vitro data from Steidler et. al as well as 14 days which was the length of Steidler's experiments. The equation for drug concentration then becomes:

$$D ( t ) = \frac{D}{V} \frac{{e}^{- ( n * ke * 2.303 * T )}}{{e}^{- ( ke * 2.303 * T )}} * {e}^{ke * 2.303 * t} * S * 14$$

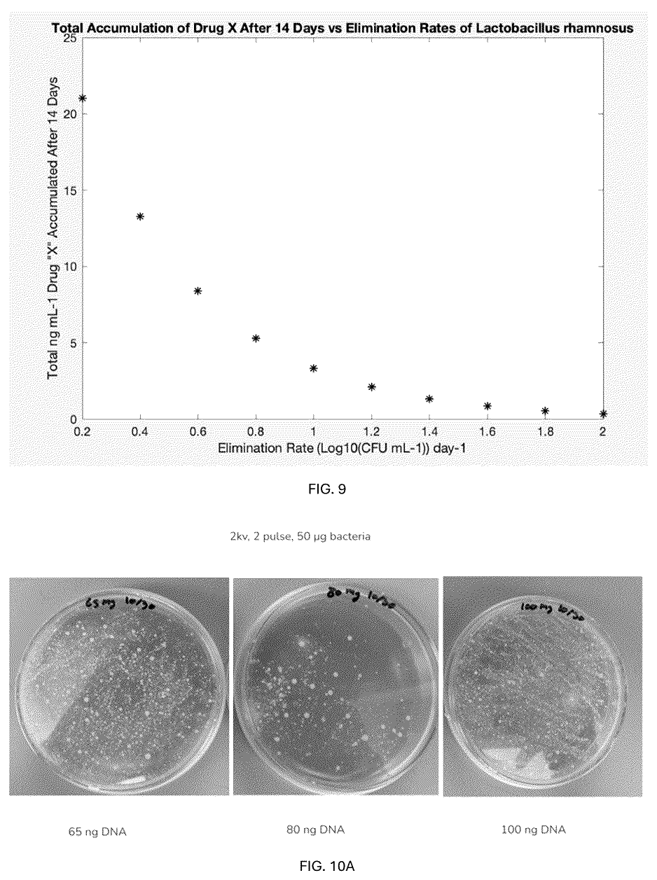

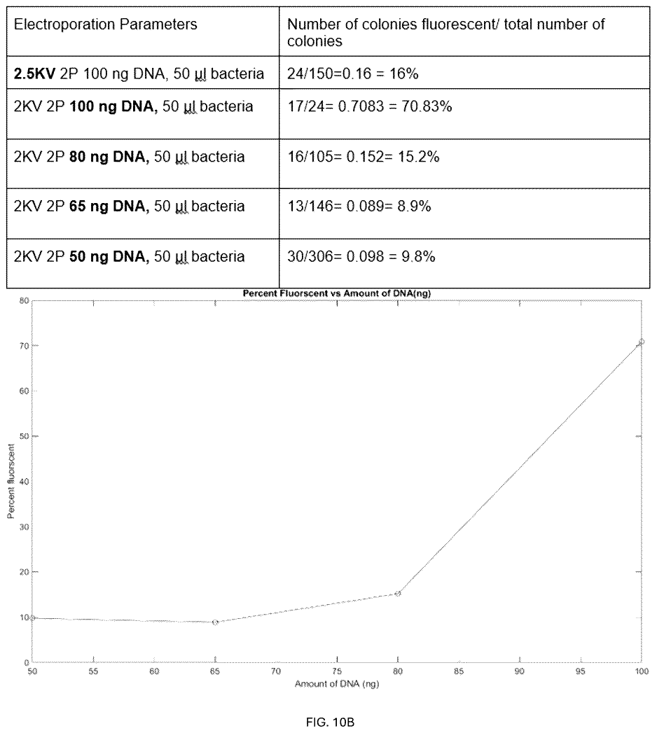

Using this equation, the concentration was plotted against elimination rates varying from 0.2 to 2 Log₁₀ (CFU mL⁻¹) day⁻¹ to reflect our fecal collection results in MATLAB. (See FIG. 9)

### Statistical Analysis

Error bars were tabulated using standard deviation of the results. Statistical analysis was done using repeated measures ANOVA through GraphPad Prism 10. Statistical significance was noted with a star, calculated as having a p<0.05. Figures that do mention statistical significance had a sample size of n=1 or did not have statistical significance.

The following Examples are provided to illustrate certain embodiments of the invention. It is not intended to limit the invention in any way.

### Example I: In Vivo Evolution of Lactobacillus rhammosus to Generate Prolonged Persistence in the Digestive Tract

Lactobacillus rhamnosus, is a renowned probiotic organism found in many food supplements. The in vivo evolution of microorganisms as a method to generate novel, non-naturally occurring bacterial strains with longer bioavailability in the intestinal tract is analyzed herein. Lactobacillus rhamnosus was autologously gavaged to mice. Subsequently, the bacteria was selected and grown ex vivo after passage through the intestinal tract to form two evolutionary variants of the bacteria. A significantly longer retention time (nearly 3×) and a slower elimination rate of the bacteria in the mouse gut was observed with each evolution. The evolutionary strains were further characterized for improved traits of gut retention such as bile salt resistance, epithelial cell binding, and genetic alterations to understand potential mechanistic hypotheses. Finally, a series of heterologous gavages were performed to determine if the increased retention of the evolutionary variants were because of animal specific host adaptations. Similar results were seen following heterologous gavages, supporting the concept that intrinsic changes to Lactobacillus occurred in evolutionary isolates that were maintained independent of the host. These findings indicate that in vivo evolution can generate probiotic strains with improved traits for gut retention as compared to the wild type.

### Results

In Vivo Evolution of Lactobacillus rhamnosus Produces Strains with Prolonged Fecal Elimination

The concept of in vivo evolution allows for genetic change to the bacteria through stressors such as acid, bile salt, and competition with commensal bacteria. Through multiple, autologous passages through the gut, evolutionary isolates with increased probiotic functionality were created.

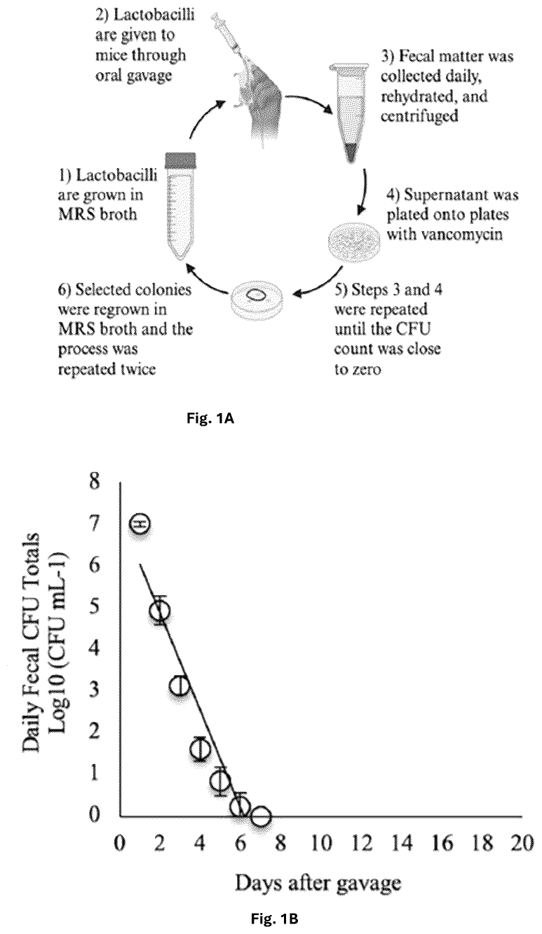

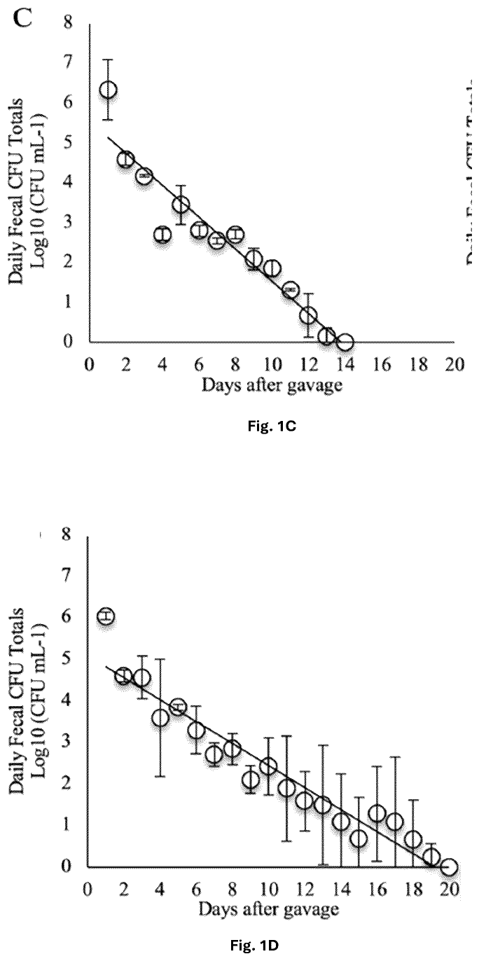

The mice were each given a single gavage containing a concentration of 0.5×10¹⁰ CFU mL⁻¹ of the wild type Lactobacillus rhamnosus. After this, fecal matter was collected daily, rehydrated and then centrifuged. The supernatant was then plated onto agar plates with vancomycin, as they were found to be naturally resistant. Samples were collected until the supernatant CFU count was less than 10. Once this low total was reached, samples were collected from the plates and then regrown. The entire gavage and collection process was repeated twice (FIG. 1A). 2 evolutions were created from the gavages. Evolution 1 is the bacteria created after the first gavage and evolution 2 is the bacteria created after the second gavage.

To visualize how the evolutions persisted in the mouse intestinal tract compared to the wild type, the Log₁₀ of the CFU mL⁻¹ totals collected from daily fecal samples were plotted. A dramatic reduction in the elimination of Lactobacilli was observed upon each evolution. In the wild-type strain, Lactobacilli were undetectable in feces by 7 days (FIG. 1B), whereas after one evolution the Lactobacilli were undetectable in feces after 14 days (FIG. 1C). After a second evolution, the Lactobacilli were undetectable in feces after 21 days (FIG. 1D), representing a 300% increase in persistence.

### Pharmacokinetic Analysis Reveals Substantial Change in Persistence of Evolutionary Strains

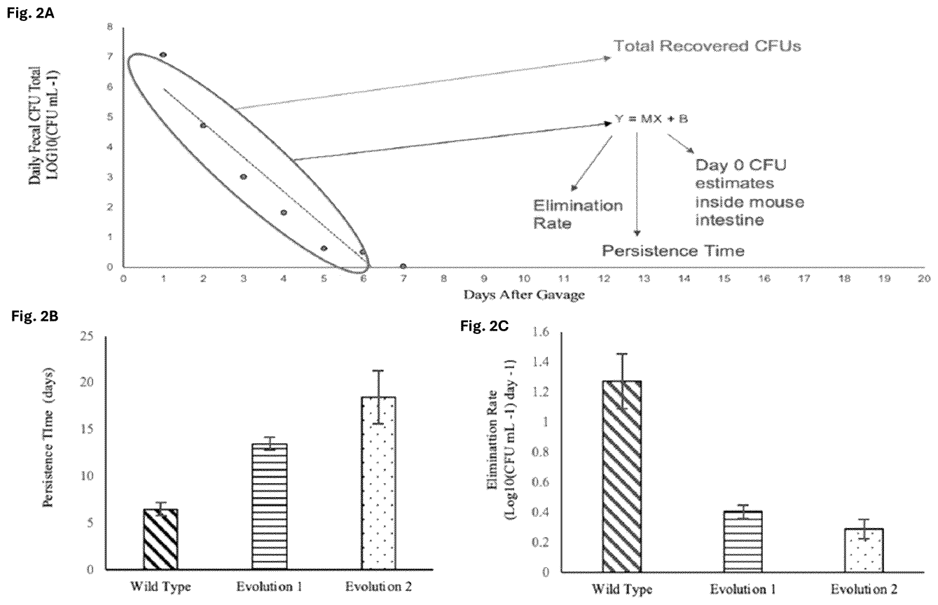

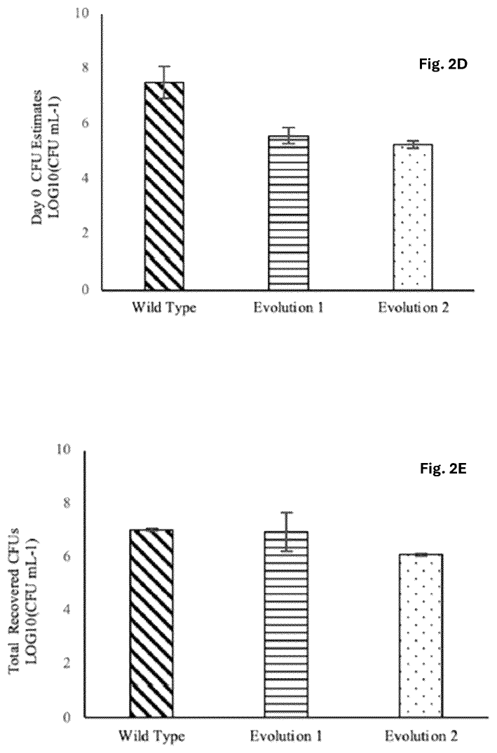

To quantitatively compare pharmacological differences between wild-type and evolutionary strains, the elimination of Lactobacilli was fit to a linear curve as outlined in FIG. 2A. These four values include persistence time, elimination rate, day 0 CFU mL⁻¹ estimates, and total CFUs recovered over the entire fecal collection period. With each evolution, the CFU persistence time increased. By evolution 2, the CFU persistence time more than tripled when compared to the wild type (FIG. 2B; an average of 6 days to 13.5 days between the wild type and evolution 1, and 18.5 days between evolutions 1 and 2). Next, elimination rates were found from the slope of the fecal collection graphs. Inversely to the persistence time, elimination rate decreased between the evolutions. There was a 74% decrease in elimination rate between the wild type and evolution 1, and a further 34% decrease between the two evolutions (FIG. 2C). Additionally, the average day 0 CFU estimates decreased between the wild type and the two evolutions with the wild type CFU being 183 times higher than that of evolution 2 (FIG. 2D). Yet, upon a subsequent evolution, the estimates for evolution 1 were ~2 times higher than those for evolution 2. Finally, the CFUs mL⁻¹ were totaled over the entire span of fecal collection for each the wild type and the two evolutions. Overall, the total recovered CFUs decreased between the wild type and the two evolutions. The wild type totals were 2.5 times higher than evolution 1, and 8.5 times higher than evolution 2 (FIG. 2E).

### Next Generation Sequencing Highlights Many Mutations in Known Persistence Genes

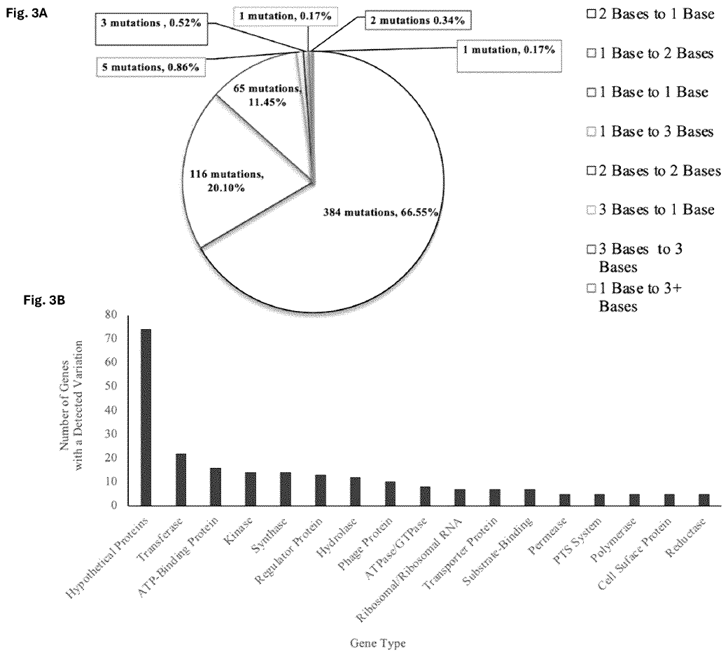

Next generation sequencing is a valuable tool that allows for the precise detection of changes between the wild type and evolutions in the genome. Cross-referencing against the deposited genome of Lactobacillus rhamnosus (NCBI database) was performed to determine which genes were altered and, therefore, may be associated with the observed probiotic persistence. From the next generation sequencing data, at a depth of 37.5, there were a total of 577 genetic variations detected, with 8 distinct types of changes (FIG. 3A). These genetic variations are disclosed in Table 1. Of those, nearly 67% of the genetic variations were two base pairs changing to one base pair. The next most common occurrence (about 20%) was the opposite where one base pair changed to two base pairs.

Furthermore, it was seen that the final 187 variations were either a change of two base pairs to one, or one base pair to two. The most significant difference is at position 842,510 where a total of 202 bases were added to the sequence. According to the genome sequence of the Lactobacillus rhamnosus, this added sequence did not happen at any specific gene locus but rather as a filler between genes [13]. It is unclear what caused this massive increase compared to the 99.9% majority which were mostly changes of three or less base pairs. Noteworthy, of the 538 detected changes, 292 of them were within a gene coding region. Of those, 197 of these changes were categorized into 17 distinct gene families, with each family having at least 5 recorded genes in that family (FIG. 3B). The most common type of gene with a detected change were hypothetical proteins with around 25% of the total detections being in genes in this category. The next most common gene type that changed were transferases, at around 8%. Additionally, 10 of the categories listed had less than 10 individual genes in that category record changes accounting for nearly 19% of the total variations. 54 exhibited more than one detected change. Out of the 56 genes, only 9 exhibited more than 2 changes, and these can be seen in FIG. 4 and Table 2.

### Evolutionary Strains Show In Vitro Resistance to Bile Salt

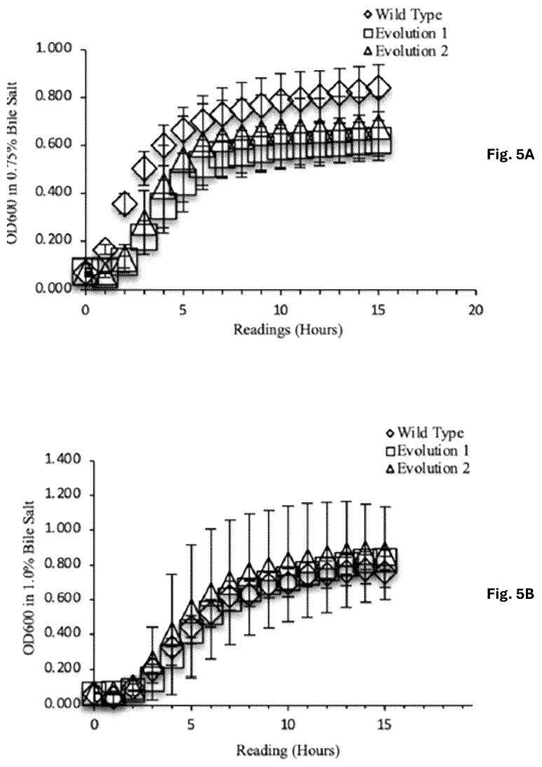

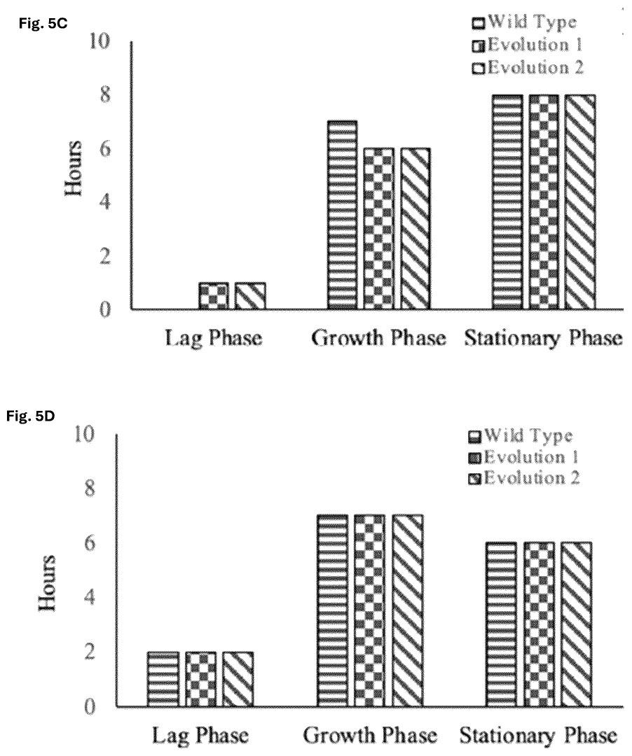

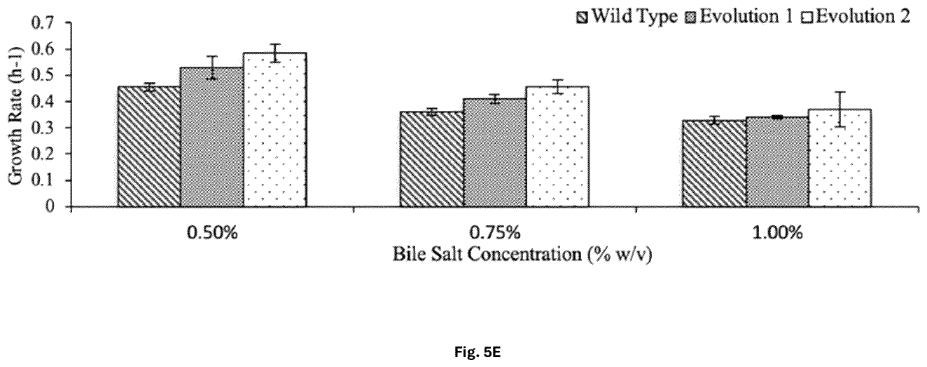

A probiotic's survival and persistence may be influenced by its resistant to bile salts which can damage the cell wall and eventually lead to cell death [14]. Genetic sequencing of evolved strains indicated that bile salt resistance pathways have been functionally altered. We, thus, tested the growth of the evolutionary isolates in various bile salt concentrations to evaluate if bacteria became bile salt resistant. The bacteria were grown in broth supplemented with bile salt at three concentrations, 0.5%, 0.75% and 1.0%. Absorbance was measured every hour over a 15-hour span. The absorbance curves were normalized to have the same starting absorbances. Results are shown with 0.5% bile salt as a representative example. The growth rates in 0.75% and 1.0% can be found in FIG. 5, as similar trends appeared in all three concentrations.

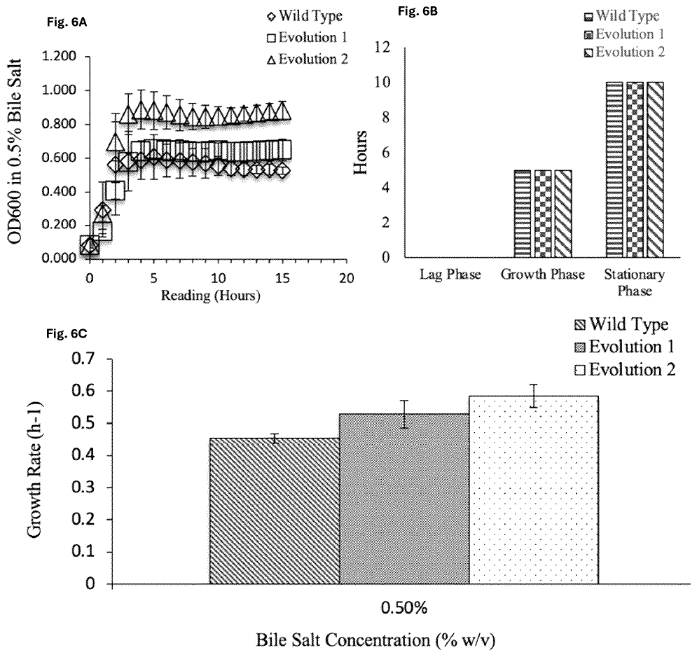

The growth patterns of the Lactobacillus in 0.50% bile salt follow the expected growth pattern of bacteria in a broth culture, including a short lag phase, before entering a growth phase that slows into a stationary phase (FIG. 6A). There was no clear lag phase present in the growth curve (FIG. 6B). Instead, there was an immediate 5-hour growth phase followed by a stationary phase for the remaining 10 hours. The growth in the other concentrations had visible lag phases in concentrations of 0.75% and 1.0% bile salt (FIG. 5A-5B). The lag phase and growth phases both increased, with increasing bile salt concentration (FIG. 5C-5D).

Further, the two evolutions had larger growth rates than the wild type: a 28% increase between evolution 2 and the wild type, and a 16% increase between evolution 1 and the wild type (FIG. 6C). Similar trends were seen with the other bile salt concentrations, in which the evolutions grew better than the wild type. Evolution 2 had the highest growth rate (FIG. 5E). The data indicated that as the bile salt concentration increased, the growth rates for all three variants decreased. Additionally, the differences between the evolutions and the wild type qualitatively became smaller.

### Reduced Caco-2 Adhesion of Evolutionary Strain Using a Perfusion Assay

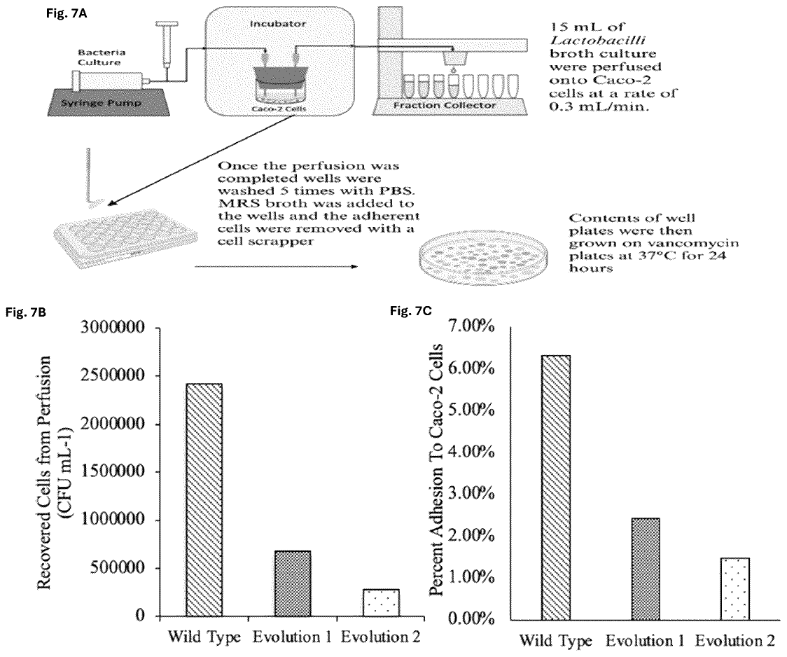

The probiotic's improved persistence time and low elimination rate could be caused by its ability to bind to the intestinal cells [16]. In order test the adhesion capability of the wild type and evolutions, a simple Caco-2 cell monolayer were used to model the intestinal epithelium [17]. Furthermore, we leveraged a perfusion system developed in our laboratory to model a slow flow of bacteria through the intestine (FIG. 7A). The bacteria were perfused onto a Caco-2 cell layer in a well plate at a rate of 0.3 mL min⁻¹. Once the perfusion was completed, the contents of the well plates were washed with PBS 5 times to remove non adherent bacteria, and the remaining cells were removed using a cell scraper and plated onto vancomycin plates. The CFU counts were then plotted for the wild type and two evolutions. Pilot studies were completed using different flow rates and Lactobacilli broth culture volumes to ensure that the perfusion system would enable adherence (data not shown). A flow rate of 0.3 mL min⁻¹ was used. Additionally, evolution 2 was arbitrarily chosen to be plated, to ensure that the cell scraper would sufficiently remove the adherent cells [11].

Overall, the total recovered CFU decreased largely between the wild type and evolution 1 and evolution 2 (FIG. 7B). This led us to reject the null hypothesis that evolved strains would bind more to an intestinal model. The CFU count was nearly 2.5 times larger in the wild type than evolution 1 and nearly 5 times larger for evolution 2. Additionally, from the CFU counts, percent adhesion was calculated. The CFU mL⁻¹ total for the broth was estimated based on a correlation between absorbance and CFU from Trabelsi et. al. Percent adhesion was calculated as the total CFUs recovered over the total perfused onto the Caco-2 cells. From the recovered CFU totals compared to the total perfused bacteria, the percent adhesion was low for all three strains, with the percent adhesion for the two evolutions both less than 3.0% (FIG. 7C). The total percent adhesion for the wild type was more than 4 times higher than evolution 2.

### Heterologous Gavages Yield Similar Results to Autologous Gavages, Indicating Non-Host Specific Adaptations

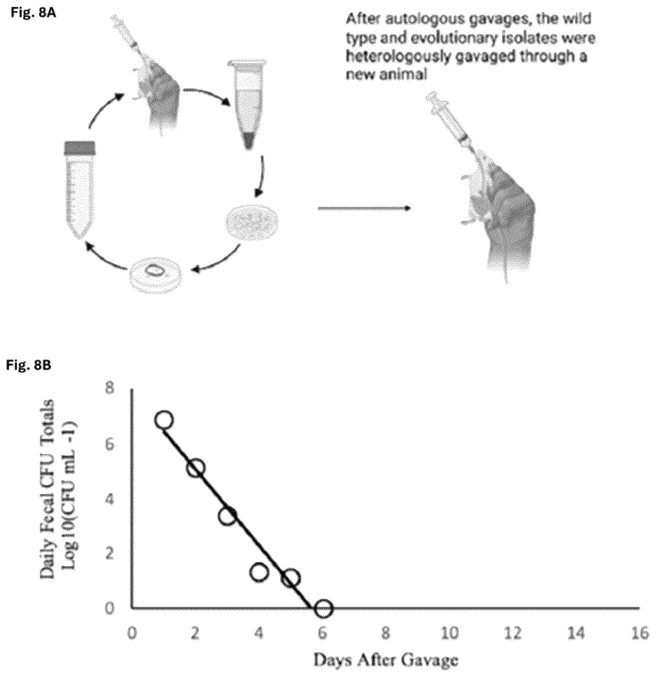

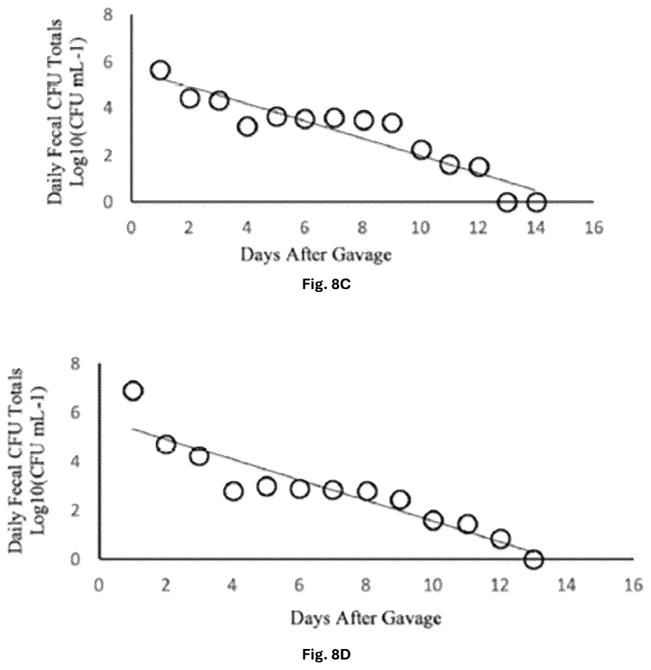

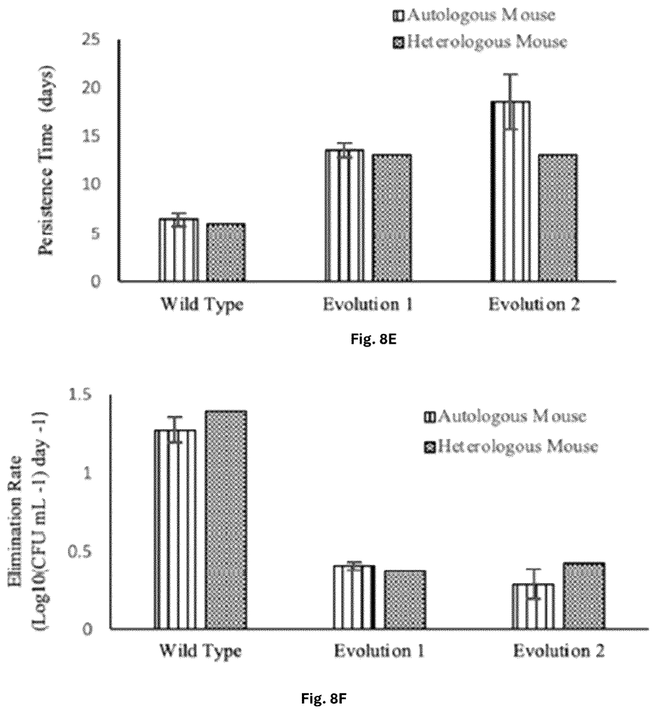

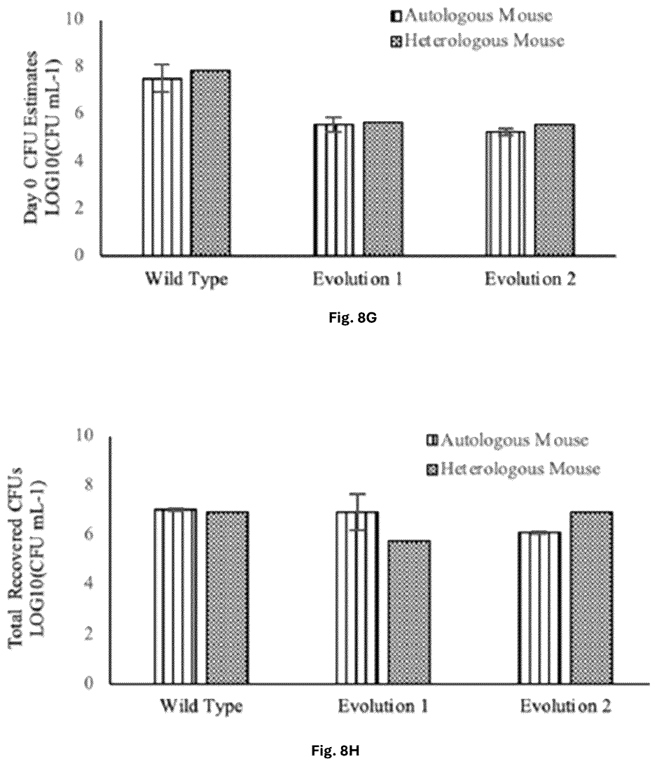

A final series of heterologous gavages were performed using a naïve animal that had not already been exposed to Lactobacilli (FIG. 8A). This study was important to determine if the changes in persistence time and elimination rate present in autologous transfers were caused by host adaptations to repeated exposure to Lactobacilli. The same data extrapolation described in FIG. 2A was used to determine how persistence and survival rates compared to the autologous data. As with the autologous gavages, the fecal matter was collected every day from the cages, and five to six pellets were rehydrated with 1 mL of deionized water.

As with the autologous gavage, the heterologous gavage of the wild type took only 6 days for the CFU count to reach zero (FIG. 8B). The heterologous gavage of evolution 1 had similar results to those seen in the autologous gavage as the CFU reached zero in approximately 13 days (FIG. 8C). However, a difference between the original autologous gavages and the heterologous gavage was seen with evolution 2. With the heterologous gavage of evolution 2, the persistence time was 13 days (FIG. 8D), which was several days shorter than the average persistence time in the autologous gavages (FIG. 8E). Parametric analysis showed that the wild type exhibited a similar elimination rate in the heterologous gavage as compared to the autologous gavages, whereas the two evolutions had much lower elimination rates in the heterologous gavages as compared to the wild type (FIG. 8F).

In the heterologous mouse, the day 0 estimates once again decreased between the wild type and the evolutions (FIG. 8G). The wild type day 0 CFUs mL⁻¹ were nearly 200 times higher than those for evolution 2, which is higher than the difference seen with the autologous gavages. Additionally, the estimated day 0 CFUs mL⁻¹ for evolution 1 are similar to those for evolution 2, only being 1.2 times higher. Overall, the estimates seen in the heterologous mouse were similar to those for the autologous gavages. The estimates for the heterologous mouse were overall higher than those for the autologous mouse, with around a 55% increase for the wild type and evolution 2, and nearly a 20% increase for evolution 1. The recovered CFUs decreased between the wild type and evolution 1, with the wild type CFUs being 14.5 times higher than evolution 1. The wild type and evolution 2 values were nearly the same, with a 2% increase in the total recovered CFUs (FIG. 8H).

### Discussion

Methods to develop more persistent probiotics are being developed for multiple applications, including longer-lasting health benefits, and long-term targeted drug delivery. Various groups have engineered probiotic bacteria to secrete anti-inflammatory cytokines as a means of treating inflammatory gastrointestinal diseases such as Inflammatory Bowel Syndrome, Ulcerative Colitis, and Crohn's Disease [19]. For instance, Steidler et al. were able to treat colitis in mice with a strain of Lactococcus lactis that was engineered to continuously secrete IL-10 in vivo [20].

Our dataset explores the effect that elimination rate has on the total accumulation of an engineered drug expressed by Lactococci. Using a one-compartment, multi-dose model, we simulated the mass transport of bacteria secreting an engineered protein in the gut, to understand the impact of elimination rate on total protein accumulation in a tissue. The methodology for this model can be found in the methods above. From this general model, which used parameters from Steidler et al. and our own study, we showed that the total accumulation of drug is highly sensitive to elimination rate. A table of parameters and their values can be found in Table 3. A change in elimination rate from 1.2 to 0.2 yields a 170× increase in drug accumulation. This basic simulation helped to justify the value of engineering improve persistence to probiotic bacteria.

TABLE 3
Defined terms in accumulation model of engineered secreted protein
expression after 14 days of daily gavages and elimination rate
of Lactobacillus rhamnosus from intestinal tract

| Term | Definition | Value and Units | Reference |
|---|---|---|---|
| D(t) | Drug ‘X’ | ng mL⁻¹ | Based on conversion of |
| | Concentration | | multidose IV-bolus |
| | after t time | | equation using bacteria |
| | | | secretion rate |
| C(t) | Bacteria | CFU mL⁻¹ | Part of multidose IV-bolus |
| | Concentration | | equation for a one |
| | after t time | | compartment model |
| | | | Bourne, 2022 |
| D | Dose | 107 CFU | Chosen reflect day 0 |
| | | | estimates from gavage |
| | | | results |
| V | Intestinal | 890 mL | Eve, 1966 Feb. 12 [37] |
| | Volume | | |
| n | Number of | 14 no unit | Part of multidose IV-bolus |
| | doses | | equation for a one |
| | | | compartment model |
| | | | Bourne, 2022 [22] |
| ke | Elimination | Varied from 0.2 to 2 | Chosen to reflect |
| | rate | Log₁₀(CFU mL⁻¹)ke | elimination rates from |
| | of bacteria | is multiplied by | gavage results. |
| | | 2.303 to convert | |
| | | from Log₁₀ | |
| | | to Ln | |
| T | Dose | Daily | Part of multidose IV-bolus |
| | Interval | | equation for a one |
| | | | compartment model |
| | | | Bourne, 2022 [22] |
| t | Time after | 0, as the model | Part of multidose IV-bolus |
| | most recent | is for 14 | equation for a one |
| | dose | days exactly | compartment model |
| | | | Bourne, 2022 [21] |
| S | Secretion | 2.5 × 10⁻⁶ ng | Steidler, L et al., 2000. [20] |
| | rate per | CFU⁻¹ day⁻¹ | |
| | day | Value is multiplied | |
| | | by 14 in the equa- | |
| | | tion to reflect | |
| | | 14 days of secretion | |

* This table describes all of the values as well as their sources used in the one-compartment, multi-dose administration model of the accumulation of bacteria in the gut.

We further investigated if repeated administration of the probiotic strains through a mouse intestinal tract could further evolve the probiotic strains to improve persistence. The initial gavage data showed that the evolutionary isolates were more persistent than the wild type. We immediately observed an increase in persistence time between the wild type and the evolutions, indicating improved survival rates within the gut. This survivability is important because, when CFU reaches zero, most, if not all of the gavaged probiotic has passed completely through the mouse intestinal tract. As seen in other literature, only daily consumption of the bacteria allowed for consistent CFU counts from fecal matter, and once ingestion of the probiotic ceased, fecal CFUs dramatically decreased [22, 23]. This also provided valuable inside regarding the overall survival of the bacteria.

With the wild type, the day 0 estimates are larger than the total recovered bacteria over the fecal collection period, indicating that more of the bacteria were killed in entering and moving through the intestinal tract than the estimates predicted. Meanwhile, for the two evolutions, the day 0 estimates were lower than the total recovered CFUs over the entire fecal collection period. This indicates that more bacteria survived throughout the gut than the fitted data estimates. However, considering that day 0 estimates and recovered totals both decrease, the evolutions most likely do not improve initial survival rates, but only improve persistence. Furthermore, the recovered CFUs are most likely a fraction of the initial inoculum.

One study from Sun et. al found that after a gavage of wild type E. coli, less than 1% of the inoculum had survived [24]. The decrease in survival is most likely due to the minimal exposure to the acid in vivo, as exposure typically lasts less than 2 hours [16]. Other studies have reported that introducing the bacteria to an acidic media does not change viability unless they are exposed for prolonged periods of time. Overall, this highlights why the persistence and elimination rate were the main parameters to improve. Much of the probiotic journey is within the small intestine and colon, where persistence factors such as colonization resistance from commensal bacteria and intestinal adhesion capability play the largest role in the survival of a probiotic [16].

Next-generation sequencing was explored and clearly showed that in vivo evolution of Lactobacillus rhamnosus led to many variations in the genome. Previous research has found that the complex survival mechanisms of probiotic bacteria are facilitated by regulator proteins, chaperone proteins, and several enzymatic proteins. Each of these proteins assist in stress resistance, metabolic function, and adherence [26]. By cross-referencing the locations of the genetic variations, determinations were made about which genes were mostly changed, as well as which genes directly impact probiotic function. Five of the altered genes encoded for cell surface proteins. These are directly responsible for the cell's ability to bind to epithelial lining [27]. Furthermore, changes in three sortase genes were observed. Sortase is key to the binding ability of the Lactobacillus rhamnosus as it helps to assemble the pili, which are key to the binding capabilities of the bacteria [26]. Changes were also detected in ATP-binding subunit genes, including those that encode for multidrug ABC transporter ATP-binding units. Similarly, all of the permeases were ABC transporters. ABC multidrug transporters could play a role in bile salt resistance by helping to pump out bile salt from the cell. This indicates that the gene alterations lead to differences in bile salt resistance [14].

Similarly, there were changes in ATPase genes, all of which are proton pumps, which aid in removing bile salt [26]. Additionally, putative glycosyltransferase BAI42722.1, one of the genes with multiple changes, helps with the creation of exopolysaccharides [26]. Exopolysaccharides have been found to contribute to the protection of the cell from harsh environmental factors such as low pH but may also lower the ability of the bacteria to bind to intestinal cells such as Caco-2 cells [28]. Finally, 46% of the genetic mutations detected were in non-coding regions. These point mutations may change the overall structure of these proteins, thereby impacting their function Additionally, using precise gene editing such as a CRISPR system, many of these mutations could be recreated to test the effects in-vitro.

Another clear quantitative difference between the wild type and the evolutions was increased bile salt resistance. One of the keys to probiotic survival and persistence is the ability to be resistant to bile salts which can damage the cell wall and eventually lead to cell death [14]. The growth rates in the bile salt concentrations were consistently higher in the evolutions than the wild type, regardless of concentration. It was seen that, at times, evolution 2 had a growth rate that was nearly double that of the wild type. The growth rates did decrease as the concentration increased. The lag and growth phases also increased to between 0.5% and 1.0%. Overall, this indicates that as the bile salt concentration increased, the time needed to become adapted to the higher stress levels increased as well [31]. If the bacteria were to be further pre-adapted to the bile salt in-vitro at lower concentrations, then larger growth rates of the bacteria at higher concentrations may occur. This combined with the multiple evolutions could allow for the future prospect of a strain that can grow well at the maximum bile concentration. The evolutions consistently maintained a higher growth rate than the wild type under bile salt conditions.

Counter-intuitively, a Caco-2 cell binding assay indicated less binding capability between the evolutions and the wild type, which would correlate with poorer probiotic capability. Many reasons could explain why the recovered CFU mL⁻¹ is much lower for the evolutions than those for the wild type. Experimentally, a low overall number of CFUs bound to Caco-2 cells which makes the methods of detection critical for accuracy. Large deviations could have occurred from initial absorbance of the broth, heterogeneity of flowing bacteria in our perfusion system, and counting of adhered bacteria. Furthermore, MRS broth may interfere with the binding capabilities of bacteria onto Caco-2 cells due to its acidity, which is why PBS is often used instead [33]. Additionally, the main binding mechanism of Lactobacillus rhamnosus to the intestinal tract is to use the pili which comprise cell surface proteins SpaABC and putative cell surface proteins SpaDEF. The pili bind both to intestinal cells but also to the mucus layer within the intestinal tract [26, 34]. Lebeer's study showed that inhibition of SpaABC limits adhesion to Caco-2 cells significantly [34]. This may explain why the evolutions did not bind as well to Caco-2 cells since the genomic sequencing showed putative mutations to the cell surface proteins. Caco-2 cells also do not contain mucus, which is a limitation to their use as a model of intestinal epithelium [17].

Finally, the results of heterologous gavage confirmed that the evolved bacteria had improved persistence over the wild type beyond the autologous gavages, as the elimination rates were lower than the elimination rate for the wild type. This indicates that the evolutionary changes from the autologous gavages were due to constant applied stressors in the intestinal tract as well as interactions with the gut microbiome, and not because of other factors (e.g., host immunity) [35]. This is critical for translation to support the use of evolved Lactobacillus rhamnosus in new hosts. One study from Duar et. al showed that different strains of Lactobacillus Reuteri derived from various hosts could survive in other species indicating that this approach is feasible in humans [36]. In conclusion, our results indicate that in vivo evolution can improve probiotic capabilities of Lactobacillus rhamnosus.

### Example II: Generation of an Evolved Probiotic Comprising a Transgene

To test whether the microorganisms described above could provide further therapeutic benefit, we further modified them to express a transgene using the following protocol. Cultures of Lactobacillus rhamnosus stored in a −80° C. freezer were inoculated for ~24 hours in MRS medium containing NaCl at a concentration of 0.7M until an optical density (OD_(600 nm)) reading of 0.8 was reached. Electroporation was carried out using a BTX electroporation system. 50 μl of competent bacteria cells were mixed with 50 to 100 ng red fluorescent protein (RFP) plasmid DNA (3-5 μl) in a 0.2-cm electrode gap cuvette and subject to the following electrical parameters: 2 KV applied voltage and 2 pulses.

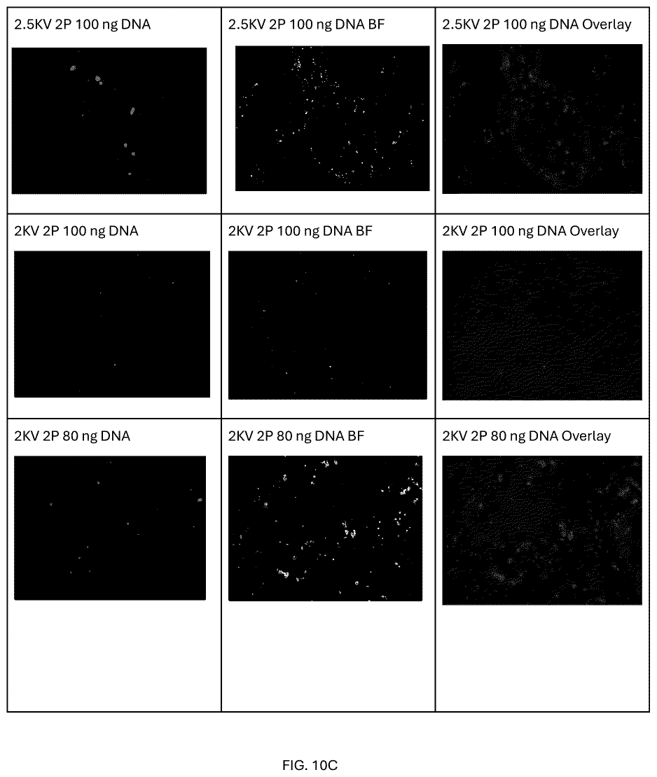

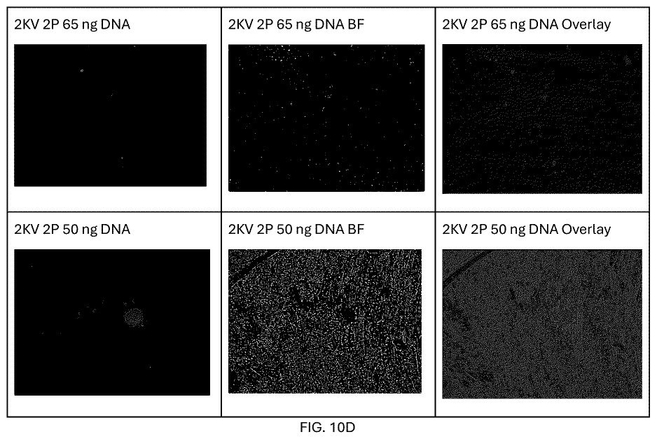

Following electroporation, cells were mixed with 0.9 ml MRS media with 0.3M sucrose and incubated at 37° C. for 4 hours to allow recovery. Afterwards, 200 μl of electroporated cells were plated on MRS agar medium plates with resistance encoded antibiotic, erythromycin (Emr). Plates were grown in an anaerobic chamber at 37° C. for 48-72 hours. Colonies were then observed under fluorescence microscopy to determine uptake of RFP plasmid DNA. (FIG. 10) This data shows that the modified microorganisms described above can be further modified to express a protein of interest, such as GLP1 (glucagon-like peptide-1), sTNFR1 (TNF receptor antagonist), IL-1RA (Interleukin-1 receptor antagonist), sGP130, EPO (erythropoietin), PTH (parathyroid hormone), insulin, or enfuvirtide and derivatives thereof.

### REFERENCES

1. Holzapfel W H, Schillinger U. Introduction to pre- and probiotics. Food Research International. 2002; 35 (2-3) 109-16 doi: 10.1016/S0963-9969 (01) 00171-5.
2. Chen J, Wang Q, Liu C, Gong J. Issues deserve attention in encapsulating probiotics: Critical review of existing literature. Critical Reviews in Food Science and Nutrition. 2017; 57 (6): 1228-1238, doi: 10.1080/10408398.2014.977991
3. Gueimonde M, Salminen S. New methods for selecting and evaluating probiotics. Digestive and Liver Disease. 2006. 38 (2) S242-S247. doi: 10.1016/S1590-8658 (07) 60003-6.
4. Mugwanda K, Hamese S, Van Zyl W F, Prinsloo E, Du Plessis M, Dicks L M T, Thimiri Govinda Raj D B. Recent advances in genetic tools for engineering probiotic lactic acid bacteria. Biosci Rep; 2023 Jan. 31; 43 (1): BSR20211299. doi: 10.1042/BSR20211299.
5. Nguyen H.-T., Truong D.-H., Kouhounde, S., Ly S.; Razafindralambo H., Delvigne F. Biochemical Engineering Approaches for Increasing Viability and Functionality of Probiotic Bacteria. Int. J. Mol. Sci. 2016; (17): 867. doi: 10.3390/ijms17060867
6. Lescat M., Launay A., Ghalayini M., Magnan M., Glodt J., Pintard C., Dion S., Denamur E. and Tenaillon, O. Using long-term experimental evolution to uncover the patterns and determinants of molecular evolution of an Escherichia coli natural isolate in the streptomycin-treated mouse gut. Mol Ecol. 2017; 26:1802-1817. doi: 10.1111/mec.13851
7. McGuire T F, Sajithlal G B, Lu J, Nicholls R D, Prochownik E V In Vivo Evolution of Tumor-Derived Endothelial Cells. PLOS ONE. 2012; 7 (5): e37138.
8. Li, W., Yao, G., Cai, H., Bai M., Kwok, L., Sun, Z., Comparative genomics of in vitro and in vivo evolution of probiotics reveals energy restriction not the main evolution driving force in short term. Genomics. 2021; 113 (5): 3373-3380, doi: 10.1016/j.ygeno.2021.07.022.
9. Campedelli I, Mathur H, Salvetti E, Clarke S, Rea M C, Torriani S, Ross R P, Hill C, O'Toole P W. Genus-Wide Assessment of Antibiotic Resistance in Lactobacillus spp. Appl Environ Microbiol. 2018 Dec. 13; 85 (1): e01738-18. doi: 10.1128/AEM.01738-18. PMID: 30366997; PMCID: PMC6293106.
10. Erickson P, Doshi A, Jetley G, Amin P, Mejevdiwala A, Patel A, Bento R, Parekkadan B. Multi-Stream Perfusion Bioreactor Integrated with Outlet Fractionation for Dynamic Cell Culture. J Vis Exp. 2022 Jul. 20; 185. doi: 10.3791/63935. PMID: 35938803; PMCID: PMC9631111.
11. Reddy S, Austin F. Adhesion and Invasion Assay Procedure Using Caco-2 Cells for Listeria monocytogenes. Bio Protoc. 2017 May 5; 7 (9): e2267. doi: 10.21769/BioProtoc.2267. PMID: 34541252; PMCID: PMC8410283.
12. Qin D. Next-generation sequencing and its clinical application. Cancer Biol Med. 2019 February; 16 (1): 4-10. doi: 10.20892/j.issn.2095-3941.2018.0055. PMID: 31119042; PMCID: PMC6528456.
13. Morita H, Toh H, Oshima K, Murakami M, Taylor T D, Igimi S, Hattori M. Complete genome sequence of the probiotic Lactobacillus rhamnosus ATCC 53103. J Bacteriol. 2009 Oct. 9; 191 (24): 7630-1. doi: 10.1128/JB.01287-09. PMID: 19820099; PMCID: PMC2786603.
14. Whitehead K, Versalovic J, Roos S, Britton R A. Genomic and genetic characterization of the bile stress response of probiotic Lactobacillus reuteri ATCC 55730. Appl Environ Microbiol. 2008 March; 74 (6): 1812-9. doi: 10.1128/AEM.02259-07. Epub 2008 Feb. 1. PMID: 18245259; PMCID: PMC2268311.
15. Hall B, Acar H, Nandipati A, Barlow M. Growth Rates Made Easy, Molecular Biology and Evolution, 2014 Jan. 31; 1:232-238, doi: 10.1093/molbev/mst187
16. Han S, Lu Y, Xie J, Fei Y, Zheng G, Wang Z, Liu J, Lv L, Ling Z, Berglund B, Yao M, Li L. Probiotic Gastrointestinal Transit and Colonization After Oral Administration: A Long Journey. Front Cell Infect Microbiol. 2021 March; 10 (11): 609722. doi: 10.3389/fcimb.2021.609722. PMID: 33791234; PMCID: PMC8006270.
17. Lea, T. Caco-2 Cell Line. In: Verhoeckx, K., et al. The Impact of Food Bioactives on Health. Springer, Cham. 2015 doi: 10.1007/978-3-319-16104-4_10
18. Trabelsi, Imen, Bejar, Wacim, Ayadi, Dorra, Chouayekh, Hichem, Kammoun, Radhouane, Bejar, Samir, Ben Salah, Riadh. Encapsulation in alginate and alginate coated-chitosan improved the survival of newly probiotic in oxgall and gastric juice. International journal of biological macromolecules. 2013; 61. doi: 10.1016/j.ijbiomac.2013.06.035.
19. Shigemori S, Shimosato T. Applications of Genetically Modified Immunobiotics with High Immunoregulatory Capacity for Treatment of Inflammatory Bowel Diseases. Front Immunol. 2017 Jan. 25; 8:22. doi: 10.3389/fimmu.2017.00022. PMID: 28179904; PMCID: PMC5263139.
20. Steidler, L et al. (2000). Treatment of Murine Colitis by Lactococcus lactis Secreting Interleukin-10. Science (289): 1352-1355 doi: 10.1126/science.289.5483.1352
21. Repeated Intravenous Bolus Doses In: Bourne, D. Basic Pharmacokinetics v1.5.5. 2022; https://sheffield.pressbooks.pub/introducingmathematicalbiology/chapter/repeated-intravenous-bolus-doses/22.
22. Wang, R., Chen, S., Jin, J., Ren, F., Li, Y., Qiao, Z., Wang, Y. and Zhao, L. Survival of Lactobacillus casei strain Shirota in the intestines of healthy Chinese adults. Microbiol Immunol, 2015; 59:268-276. doi: 10.1111/1348-0421.12249
23. Tuohy, K. M., Pinart-Gilberga, M., Jones, M., Hoyles, L., McCartney, A. L. and Gibson, G. R. Survivability of a probiotic Lactobacillus casei in the gastrointestinal tract of healthy human volunteers and its impact on the faecal microflora. Journal of Applied Microbiology, 2007; 102:1026-1032. doi: 10.1111/j.1365-2672.2006.03154.x
24. Sun, F. J., Kaur, S., Ziemer, D. et al. Decreased Gastric Bacterial Killing and Up-Regulation of Protective Genes in Small Intestine in Gastrin-Deficient Mouse. Dig Dis Sci 2003; 48:976-985 doi: 10.1023/A: 1023068116934
25. Sanhueza, E., Paredes-Osses, E., Gonzalez, C., Garcia, A. Effect of pH in the survival of Lactobacillus salivarius strain UCO_979C wild type and the pH acid acclimated variant. Electron. J. Biotechnol. 2003; 18 (5): 343-346. doi: 10.1016/j.ejbt.2015.06.005
26. Lebeer S, Vanderleyden J, De Keersmaecker S C. Genes and molecules of lactobacilli supporting probiotic action. Microbiol Mol Biol Rev. 2008 December; 72 (4): 728-64. doi: 10.1128/MMBR.00017-08. PMID: 19052326; PMCID: PMC2593565.
27. Kankainen M, Paulin L, Tynkkynen S, von Ossowski I, Reunanen J, Partanen P, Satokari R, Vesterlund S, Hendrickx A P, Lebeer S, De Keersmaecker S C, Vanderleyden J, Hämäläinen T, Laukkanen S, Salovuori N, Ritari J, Alatalo E, Korpela R, Mattila Sandholm T, Lassig A, Hatakka K, Kinnunen K T, Karjalainen H, Saxelin M, Laakso K, Surakka A, Palva A, Salusjärvi T, Auvinen P, de Vos W M. Comparative genomic analysis of Lactobacillus rhamnosus GG reveals pili containing a human-mucus binding protein. Proc Natl Acad Sci USA. 2009 Sep. 17; 106 (40): 17193-8. doi: 10.1073/pnas.0908876106. Epub PMID: 19805152; PMCID: PMC2746127.
28. Castro-Bravo N, Wells J M, Margolles A and Ruas-Madiedo P. Interactions of Surface Exopolysaccharides From Bifidobacterium and Lactobacillus Within the Intestinal Environment. Front. Microbiol. 2018; 9:2426. doi: 10.3389/fmicb.2018.02426
29. Dehghanpoor R, Ricks E, Hursh K, Gunderson S, Farhoodi R, Haspel N, Hutchinson B, Jagodzinski F. Predicting the Effect of Single and Multiple Mutations on Protein Structural Stability. Molecules.; 2018; 23 (2): 251. doi: 10.3390/molecules23020251
30. Rees H A, Liu D R. Base editing: precision chemistry on the genome and transcriptome of living cells. Nat Rev Genet. 2018 Oct. 19; 19 (12): 770-788. doi: 10.1038/s41576-018-0059-1. PMID: 30323312; PMCID: PMC6535181.
31. Bertrand R L. Lag Phase Is a Dynamic, Organized, Adaptive, and Evolvable Period That Prepares Bacteria for Cell Division. J Bacteriol. 2019; 201 doi: 10.1128/jb.00697-18.
32. Kristoffersen S M, Ravnum S, Tourasse N J, Økstad O A, Kolstø A B, Davies W. Low concentrations of bile salts induce stress responses and reduce motility in Bacillus cereus ATCC 14579 [corrected]. J Bacteriol. 2019; 189 (14): 5302-13. doi: 10.1128/JB.00239-07.
33. Lehto E., Seppo S, Inhibition of Salmonella typhimurium adhesion to Caco-2 cell cultures by Lactobacillus strain GG spent culture supernate: only a pH effect?. FEMS Immunology & Medical Microbiology. June 1997; 18 (2): 125-132, doi: 10.1111/j. 1574-695X.1997.tb01037.x
34. Lebeer S, Claes I, Tytgat H L, Verhoeven T L, Marien E, von Ossowski I, Reunanen J, Palva A, Vos W M, Keersmaecker S C, Vanderleyden J. Functional analysis of Lactobacillus rhamnosus GG pili in relation to adhesion and immunomodulatory interactions with intestinal epithelial cells. Appl Environ Microbiol. 2012 January; 78 (1): 185-93. doi: 10.1128/AEM.06192-11. PMID: 22020518; PMCID: PMC3255643.
35. Jiang S, Zhang C, Han Z, Ma W, Wang S, Huo D, Zhai Q, Huang S, Zhang J. Native microbiome dominates over host factors in shaping the probiotic genetic evolution in the gut. npj Biofilms Microbiomes 2023; 9 (80) doi: 10.1038/s41522-023-00447-8
36. Duar RMFrese SALin XBFernando S C, Burkey T E, Tasseva G, Peterson DABlom J, Wenzel C Q, Szymanski C M, Walter J Experimental Evaluation of Host Adaptation of Lactobacillus reuteri to Different Vertebrate Species. Appl Environ Microbiol 2017; 83: e00132-17. doi: 10.1128/AEM.00132-17
37. Eve I S. A review of the physiology of the gastrointestinal tract in relation to radiation doses from radioactive materials. Health Phys. 1966 Feb. 12; (2): 131-61. p.147 doi: 10.1097/00004032-196602000-00002

### Embodiments

Embodiment 1. A method for producing microorganisms with enhanced gut persistence, the method comprising:
a) administering a microorganism to a subject,
b) collecting the fecal matter from the subject, and
c) separating a modified microorganism from the fecal matter, said modified microorganism having enhanced gut persistence.

Embodiment 2. The method of embodiment 1, wherein the modified microorganism has a decreased elimination rate and/or increased bile salt resistance.

Embodiment 3. The method of embodiment 1 or embodiment 2, wherein the fecal matter is collected at least one day after administering the microorganism.

Embodiment 4. The method of any one of the preceding embodiments, further comprising, ex vivo culturing of the microorganism after separation from the fecal matter.

Embodiment 5. The method of any one of the preceding embodiments, further comprising repeating steps a)-c) with the modified microorganism at least one time.

Embodiment 6. The method of any one of the preceding embodiments, wherein the microorganism is selected from L. rhamnosus, L. acidophilus, L. plantarum, L. rhamnosus, L. reuteri, L. casei, L. delbrueckii subsp. Bulgaricus, L. gasseri, L. fermentum, L. johnsonii, L. paracasei, L. salivarius, B. bifidum, B. animalis, B. breve, B. longum, B. adolescentis, B. infantis, Saccharomyces boulardii and Saccharomyces cerevisiae.

Embodiment 7. The method of embodiment 6, wherein the microorganism is L. rhamnosus.

Embodiment 8. The method of any one of the preceding embodiments, wherein the subject is a human or a mouse.

Embodiment 9. The method of any one of the preceding embodiments, wherein the modified microorganism has a gut persistence that is at least 100% greater, at least 200% greater, or at least 300% greater than a control unmodified organism.

Embodiment 10. The method of any one of the preceding embodiments, wherein the modified microorganism has at least one persistence-associated genetic modification.

Embodiment 11. The method of embodiment 10, wherein the at least one persistence-associated genetic modification is selected from the genetic modifications listed in Table 1 or is present in the genes listed in FIGS. 3B, 4, or Table 2.

Embodiment 12. A microorganism produced by the method of any one of the preceding embodiments.

Embodiment 13. A probiotic formulation comprising the microorganism of embodiment 12.

Embodiment 14. A probiotic formulation comprising at least one microorganism having at least one persistence-associated genetic modification.

Embodiment 15. The probiotic formulation of embodiment 14, wherein the at least one persistence-associated genetic modification is selected from the genetic modifications listed in Table 1 or is present in the genes listed in FIGS. 3B and 4.

Embodiment 16. The probiotic formulation of embodiment 15, wherein the microorganism has each of the persistence-associated genetic modifications present in the genes listed in FIGS. 3B and 4 or each of the genetic modifications listed in Table 1.

Embodiment 17. The probiotic formulation of any one of embodiments 14-16, wherein the modified microorganism has a gut persistence that is at least 100% greater, at least 200% greater, or at least 300% greater than a control.

Embodiment 18. The probiotic formulation of any one of embodiments 13-17, wherein the microorganism comprises a transgene.

Embodiment 19. The probiotic formulation of embodiment 18, wherein the transgene is selected from, GLP1, sTNFR1, IL-1RA, sGP130, EPO, PTH, insulin, or enfuvirtide and derivatives thereof.

Embodiment 20. The probiotic formulation of any one of embodiments 13-19, further comprising at least one formulary ingredient.

Embodiment 21. The probiotic formulation of any one of embodiments 13-20, wherein the microorganism is present in a concentration of about 1×10⁶ CFU to about 1×10¹² CFU. The probiotic formulation of any one of embodiments 13-21, further Embodiment 22. comprising at least one anti-inflammatory agent.

Embodiment 23. The probiotic formulation of embodiment 22, wherein the anti-inflammatory agent is selected from corticosteroids, aspirin, celecoxib, diclofenac, diflunisal, etodolac, ibuprofen, indomethacin, ketoprofen, ketorolac, nabumetone, naproxen, oxaprozin, piroxicam, salsalate, sulindac, tolmetin, interleukin (IL)-1 receptor antagonist, IL-4, IL-6, IL-10, IL-11, IL-13, cytokine receptors for IL-1, tumor necrosis factor-alpha, IL-18 and derivatives and biosimilars thereof. Examples of inflammatory bowel disease therapeutics include, without limitation: anti-inflammatory agents, a TNF alpha inhibitor, monoclonal antibodies, a receptor fusion protein, steroids, aminosalicylates, and immunomodulators or immunosuppressants.

Embodiment 24. A method of treating an inflammatory disease comprising administering the probiotic formulation of any one of embodiments 13-23.

Embodiment 25. The method of embodiment 24, further comprising administering at least one anti-inflammatory agent.

Embodiment 26. The method of embodiment 25, wherein the anti-inflammatory agent is selected from corticosteroids, aspirin, celecoxib, diclofenac, diflunisal, etodolac, ibuprofen, indomethacin, ketoprofen, ketorolac, nabumetone, naproxen, oxaprozin, piroxicam, salsalate, sulindac, tolmetin, interleukin (IL)-1 receptor antagonist, IL-4, IL-6, IL-10, IL-11, IL-13, cytokine receptors for IL-1, tumor necrosis factor-alpha, IL-18 and derivatives and biosimilars thereof. Examples of inflammatory bowel disease therapeutics include, without limitation: anti-inflammatory agents, a TNF alpha inhibitor, monoclonal antibodies, a receptor fusion protein, steroids, aminosalicylates, and immunomodulators or immunosuppressants.

Embodiment 27. The method of any one of embodiments 24-26, wherein the inflammatory disease is selected from Crohn's Disease, ulcerative colitis, indeterminate colitis, lymphocytic colitis, ischaemic colitis, diversion colitis, microscopic colitis, infective colitis, collagenous colitis, Bahcet's syndrome, idiopathic inflammation of the small and/or proximal intestine, and IBD-related diarrhea.

Embodiment 28. The method of any one of embodiments 24-27 wherein administering the probiotic composition ameliorates symptoms of the disease.

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

What is claimed is:

1. A method for producing microorganisms with enhanced gut persistence, the method comprising:
a) administering a microorganism to a subject,
b) collecting the fecal matter from the subject, and
c) separating a modified microorganism from the fecal matter,
said modified microorganism having enhanced gut persistence.

2. The method of claim 1, wherein the modified microorganism has a decreased elimination rate and/or increased bile salt resistance.

3. The method of claim 1, wherein the fecal matter is collected at least one day after administering the microorganism.

4. The method of claim 1, further comprising at least one of:
a) ex vivo culturing of the microorganism after separation from the fecal matter, and
b) repeating steps a)-c) with the modified microorganism at least one time.

5. The method of claim 1, wherein the microorganism is selected from L. rhamnosus, L. acidophilus, L. plantarum, L. rhamnosus, L. reuteri, L. casei, L. delbrueckii subsp. Bulgaricus, L. gasseri, L. fermentum, L. johnsonii, L. paracasei, L. salivarius, B. bifidum, B. animalis, B. breve, B. longum, B. adolescentis, B. infantis, Saccharomyces boulardii and Saccharomyces cerevisiae.

6. The method of claim 4, wherein the microorganism is L. rhamnosus.

7. The method of claim 1, wherein the modified microorganism has at least one of a) a gut persistence that is at least 100% greater, at least 200% greater, or at least 300% greater than a control unmodified organism, and b) at least one persistence-associated genetic modification.

8. The method of claim 7, wherein the at least one persistence-associated genetic modification is selected from the genetic modifications listed in Table 1 or is present in the genes listed in FIGS. 3B, 4, or Table 2.

9. A microorganism produced by the method of claim 1.

10. A probiotic formulation comprising a microorganism produced by the method of claim 1.

11. A probiotic formulation comprising at least one microorganism having at least one persistence-associated genetic modification.

12. The probiotic formulation of claim 11, wherein the at least one persistence-associated genetic modification is selected from the genetic modifications listed in Table 1 or is present in the genes listed in FIGS. 3B and 4.

13. The probiotic formulation of claim 12, wherein the microorganism has each of the persistence-associated genetic modifications present in the genes listed in FIGS. 3B and 4 or each of the genetic modifications listed in Table 1.

14. The probiotic formulation of claim 10, wherein the modified microorganism has a gut persistence that is at least 100% greater, at least 200% greater, or at least 300% greater than a control.

14. The probiotic formulation of claim 10, wherein the microorganism comprises a transgene.

15. The probiotic formulation of claim 14, wherein the transgene is selected from, GLP1, STNFR1, IL-1RA, sGP130, EPO, PTH, insulin, or enfuvirtide and derivatives thereof.

16. The probiotic formulation of claim 10, further comprising at least one formulary ingredient and/or at least one anti-inflammatory agent.

17. The probiotic formulation of claim 16, wherein the anti-inflammatory agent is selected from corticosteroids, aspirin, celecoxib, diclofenac, diflunisal, etodolac, ibuprofen, indomethacin, ketoprofen, ketorolac, nabumetone, naproxen, oxaprozin, piroxicam, salsalate, sulindac, tolmetin, interleukin (IL)-1 receptor antagonist, IL-4, IL-6, IL-10, IL-11, IL-13, cytokine receptors for IL-1, tumor necrosis factor-alpha, IL-18 and derivatives and biosimilars thereof. Examples of inflammatory bowel disease therapeutics include, without limitation: anti-inflammatory agents, a TNF alpha inhibitor, monoclonal antibodies, a receptor fusion protein, steroids, aminosalicylates, and immunomodulators or immunosuppressants.

18. The probiotic formulation of claim 10, wherein the microorganism is present in a concentration of about 1×10⁶ CFU to about 1×10¹² CFU.

19. A method of treating an inflammatory disease comprising administering the probiotic formulation of claim 10.

20. The method of claim 19, wherein the inflammatory disease is selected from Crohn's Disease, ulcerative colitis, indeterminate colitis, lymphocytic colitis, ischaemic colitis, diversion colitis, microscopic colitis, infective colitis, collagenous colitis, Bahcet's syndrome, idiopathic inflammation of the small and/or proximal intestine, and IBD-related diarrhea.
